(19) United States
(12) Patent Application Publication
(10) Pub. No.: US 2020/0330378 A1
(43) Pub. Date: Oct. 22, 2020

(54) TASTE-ENHANCED CANNABINOID SUBMICRON EMULSION SYRUP COMPOSITIONS

(71) Applicant: ICDPHARMA LTD., Carme-Yosef (IL)

(72) Inventor: Doron FRIEDMAN, Carme-Yosef (IL)

(21) Appl. No.: 16/959,899

(22) PCT Filed: Jan. 2, 2019
(86) PCT No.: PCT/IL2019/050008

§ 371 Date: Jul. 2, 2020

Related U.S. Application Data
(60) Provisional application No. 62/613,194, filed on Jan. 3, 2018.

(51) Int. Cl. A61K 9/00; A61K 9/107; A61K 47/26; A61K 47/44; A61K 31/05

(52) U.S. Cl. CPC A61K 9/0095; A61K 9/1075; A61K 45/06; A61K 47/44; A61K 31/05; A61K 47/26

(57) ABSTRACT

The present invention provides a stable oil-in-water submicron emulsion oral cannabinoid composition, comprising at least one cannabinoid in a pharmaceutically acceptable carrier, at least one triglyceride oil, at least two emulsifiers, at least one taste-enhancing excipient and water, wherein the composition is essentially free of bitter taste, and is essentially free of phospholipids, liposomes and/or micelles. The present invention also encompasses methods and a kit based on said composition.

### CROSS REFERENCE TO RELATED APPLICATIONS

This application claims priority to U.S. Provisional Patent Application Ser. No. 62/613,194, filed on Jan. 3, 2018, the entire contents of which are hereby incorporated by reference in its entirety.

### FIELD OF THE INVENTION

The present invention relates to a stable emulsion composition of oil-in-water type, comprising at least one hydrophobic cannabinoid with low bioavailability in a pharmaceutically acceptable carrier, wherein the composition is emulsified to sub-micron particles or nanoparticles, the at least one cannabinoid is essentially solubilized in the oil inner phase and water is in the outer phase. The stable emulsion composition exhibits enhanced oral bioavailability and solubility and provides a convenient method of administration and lower dosages of the at least one cannabinoid and not any bitter after taste associated with solubilized cannabinoids.

### BACKGROUND OF THE INVENTION

Cannabis extracts comprise hundreds of identified phyto-chemicals, however two distinct groups of bio-active molecules are responsible for their therapeutic activity, the cannabinoids and the terpenes. The most important and abundant Cannabis terpenes and terpenoids are: limonene, myrcene, α-pinene, linalool, β-caryophyllene, caryophyllene oxide, nerolidol and phytol. Most terpenoids are flavor and fragrance components common to human diets that have been designated Generally Recognized as Safe (GRAS) by the US Food and Drug Administration and other regulatory agencies. Terpenoids are quite potent and affect animal and even human behavior when inhaled from ambient air at serum levels in the single digits ng/mL, as they exhibit unique therapeutic effects.

Common Cannabis plants are described as C. sativa or C. indica and are believed to produce different consumer experience and pharmacological effects which are solely related to the cannabinoid composition. Some terpenes or terpenes compositions are more alerting, and some are more sedative.

U.S. Pat. No. 6,383,513 Watts at al., describes an oil-in-water type emulsion which I a nasal delivery, produced by high pressure homogenization, and stabilized with phospholipids. The U.S. Pat. No. 6,383,513 patent does not provide information on taste masking or stability.

In United States Patent Application 20180042845 (to Sinai at al.), “Preparations of cannabis emulsions and methods thereof”, in the composition of claim 1, the emulsion particle size is in the range of about 100 nm to about 400 nm. However, the stability is limited to maximum 12 months (the composition is stable at room temperature for about 3 months to about 12 months) and it is of high oil content.

U.S. Pat. No. 9,907,823 (Kuhrts) “Methods and formulations for increasing the water solubility and/or bioavailability of a phyto-cannabinoid compound is disclosed herein. In one example, a water-soluble phyto-cannabinoid formulation can comprise a phyto-cannabinoid; a non-ionic surfactant; and optionally, water. The weight ratio of phyto-cannabinoid content to non-ionic surfactant can be from 1:10,000 to 1:5.” Such very high rations are typically producing micelles and not emulsions, in contrast to current invention whereas the ratios are significantly different and typical for emulsions systems of oil-in-water.

United States Patent Application 20170348276 (Bryson, at al) A nasally administered cannabinoid semi-solid or viscous liquid composition, provides a non-oral, non-injectable form of cannabinoids. The composition do not comprise sweetener and or taste masking and therefore is not appropriate for oral administration and also no stability or particle size are provided and the oily internal phase is exceeding 10% of the composition.

Emulsions are thermodynamically unstable compositions, which tend to separate over time. The emulsion separation is governed by the Stocks equation, and it is mainly a function of the particle size and the medium viscosity.

There is an unmet need for ready to use and patient-friendly oral cannabinoids dosage forms for the various populations, children, adults and elderly, particularly for those that are suffering from dysphagia and those that are struggling to swallow hard dosage forms such as tablets or capsules, wherein the dosage form will mask the unpleasant bitter after taste of solubilized cannabinoids and will comprise the required cannabinoids and optionally terpenes or essential oils to maximize pharmacological efficacy and that will improve the poor oral bioavailability the cannabinoids. In addition, there is unmet need for cannabis oral medication with specific pharmacological effects such as increased alertness and sedative as well as for treatment of insomnia, depression, fatigue and pain and for improving wellbeing in elderly patients in highly acceptable and convenient dosage forms that will contribute to maximal patent compliance.

### SUMMARY OF THE INVENTION

The present invention relates to stable compositions, formulations and delivery systems, for the administration of combinations of at least one cannabinoid emulsified oil-in-water type sub-micron emulsion system that provides improved oral absorption and devoid of bitter after taste. More particularly, the present invention relates to compositions and dosage forms of cannabinoids and emulsifiers, which form an oil-in-water type emulsion, having a plurality of particles in the sub-micron or nanoparticle range, improved oral bioavailability through the gastrointestinal tract, much reduced bitter taste and after taste and long shelf life stability.

In some embodiments, there is provided a stable emulsion composition comprising from about 0.05% w/w to about 10% w/w of at least one cannabinoid in a pharmaceutically acceptable carrier, from about 1% w/w to about 10% w/w of at least one triglyceride oil, from about 1% w/w to about 10% w/w of at least two emulsifiers, of which one having an HLB value from about 10 to about 16 and the other(s) having an HLB from about 2 to about 8, from about 1% w/w to about 40% w/w of at least one taste-enhancing excipient and from about 1% w/w to about 60% w/w of water. wherein the emulsion composition is a plurality of sub-micron or nano-particles.

wherein the emulsion composition is stable at ambient temperature for at least 12-24 months
wherein the emulsion composition does not separate or cream under accelerated conditions

a. at 40° C. for at least three months and

b. under centrifugation of about 3,500 RPM for about 5 minutes.

wherein the composition is essentially free of bitter taste, and
wherein the ratio between the at least one cannabinoid and the at least two emulsifiers is at least 1:5, and
wherein the emulsion oil phase comprises not more than 10% by weight of the composition. and
wherein the composition is essentially free of phospholipids, liposomes and/or micelles. wherein the at least one cannabinoid is essentially solubilized in the oil inner phase and water is in the principle solvent and outer or external phase of the emulsion and optionally from about 0.01% w/w to about 3% w/w of at least one terpene or essential oil

In a preferred embodiment, the ratio of the at least one cannabinoid to the at least one triglyceride oil is from about 1:5 to about 5:1, or from 1:2 to about 2:1, whereas the total cannabinoids amount by weight is above its solubility in the oil, and the wherein the ratio by weight of the at least one cannabinoid and the ratio of the at least one triglyceride oil to the at least two emulsifiers is from 1:2 to about 10:1 and wherein the total cannabinoids amount by weight is above its solubility in the oil.

In some additional embodiments, there is provided the above composition of this invention, wherein the at least one cannabinoid is in a concentration of from about 0.01% w/w to about 10% w/w, preferably 0.05% w/w to about 5% w/w, preferably from about 0.2% w/w to about 4% w/w and selected from the group consisting of a natural or synthetic cannabinoid or cannabinoid combination: selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof, cannabis oil, cannabis extract and combinations thereof.

According to some embodiments, there is provided the above composition of this invention, wherein the at least one terpene, essential oil or extract is selected from the group consisting of an essentially pure terpene or terpenoid, an essential oil selected from a Cannabis species, Allspice Berry, Amber Essence, Anise Seed, Mica, Balsam of Peru, Basil, Bay Leaf, Benzoin Gum, Bergamot, Bois de Rose (Rosewood), Cajuput, Calendula (Marigold pot), White Camphor, Caraway Seed, Cardamone, Carrot Seed, Cedarwood, Celery, German or Hungarian Chamomile, Roman or English Chamomile, Cinnamon, Citronella, Clary Sage, Clovebud, Coriander, Cumin, Cypress, Eucalyptus, Fennel, Siberian Fir needle, Frankincense (Olibanum oil), Garlic, Rose Geranium, Ginger, Grapefruit, Hyssop, Jasmine Absolute, Jojoba, Juniper Berry, Lavender, Lemon, Lemongrass, Lime, Sweet Marjoram, Mugwort, Mullein Flower, Myrrh Gum, Bigarade Neroli, Nutmeg, Bitter Orange, Sweet Orange, Oregano, Patchouly, Pennyroyal, Black Pepper, Peppermint, Petitegrain, Pine Needle, Poke Root, Rose Absolute, Rosehip Seed, Rosemary, Dalmatian Sage, Sandalwood Oil, Sassafras, Spearmint, Spikenard, Spruce (Hemlock), Tangerine, Tea Tree, Thuja (Cedar leaf), Thyme, Vanilla extract, Vetivert, Wintergreen, an extract selected from Witch Hazel (Hamamelia) Extract, and Ylang Ylang (Cananga) Extract and combinations thereof, or an essentially pure isolated terpene selected from bisabolol, borneol, caryophyllene, carene, camphene, cineol, citronella, eucalyptol, geraniol, guaiol, humulene, isopropyltoluene, isopulegol, linalool, limonene, methyl salicylate, menthol, myrcene, nerolidol, ocimene, pinene, phytol, pulegone, terpinene, terpinolene, thymol and combinations thereof.

According to some other embodiments, there is provided the above composition of this invention, wherein the at least one triglyceride oil is selected from the group consisting of vegetable oil, corn oil, peppermint oil, canola oil, poppy seed oil, palm oil, soybean oil, hydrogenated soybean oil, sesame oil, rapeseed oil, sunflower seed oil, peanut oil, castor oil, hydrogenated castor oil, cottonseed oil, palm kernel oil, coconut oil, olive oil, borage oil and combinations thereof.

In some embodiments, there is provided the above composition of this invention, wherein one of the at least one hydrophilic emulsifiers is selected from the group consisting of sucrose ester, polysorbate, polyoxyl hydrogenated castor oil, tocopherol polyethylene glycol 1000 succinate, polyoxyl glycerides, polyglyceryl fatty acid ester, and polymeric emulsifier Acrylates/C10-30 Alkyl Acrylate Cross-Polymer and combinations thereof.

In some other embodiments, there is provided the above composition of this invention, wherein one of the at least two emulsifiers having an HLB value from 10 to 16 is selected from the group consisting of sucrose fatty acid esters, sucrose stearate, sucrose distearate, sucrose palmitate, sucrose laurate sucrose polystearate and combinations thereof and the second with HLB of about 2 to about 8 selected from sorbitan fatty acid esters, polyglyceryl fatty acid esters and Polyoxyethylene fatty ether and esters.

In some additional embodiments, there is provided the above composition of this invention, wherein the at least one cannabinoid exhibits an improved oral bioavailability at least about 100% higher than the oral bioavailability of same cannabinoid, when dissolved in an organic solvent or a mixture of organic solvents.

In some embodiment the at least one surfactant with HLB of about 2 to about 8 is selected from polyglyceryl-3 dioleate HLB=3, span 80 HLB=4.3, span 85 HLB=1.8, span 60 HLB=4.7, span 40 HLB=6.7, span 83 HLB=3.7, span20=8.6, Sorbitan Oleate HLB=4.3, Sorbitan Monostearate NF HLB=4.7, Sorbitan Stearate HLB=4.7, Sorbitan Isostearate HLB=4.7, Steareth-2 HLB=4.9, Oleth-2 HLB=4.9, Glyceryl Laurate HLB=5.2 Ceteth-2 HLB=5.3,

According to some embodiments, there is provided the above composition of this invention, wherein the at least one cannabinoid exhibits an improved oral bioavailability at least from about 100% to about 200% higher than the bioavailability of same cannabinoid, when dissolved in an organic solvent or mixture of organic solvents.

According to some other embodiments, there is provided the above composition of this invention, wherein the at least one cannabinoid exhibits an oral bioavailability which is at least from about 200% to about 300% higher than the bioavailability of same at least one cannabinoid, when dissolved in organic solvent or mixture of organic solvents.

In some embodiments, there is provided the above composition of this invention, wherein at least 90%, at least 95%, at least 98% or at least 99% of the particle plurality in the stable emulsion are below a mean particle size of about 1,000 nm, preferably below about 800 nm, below about 600 nm, below about 400 nm or below about 200 nm.

In some other embodiments, there is provided the above composition of this invention, wherein the assay of the at least one cannabinoid in the composition remains essentially unchanged at ambient temperature for at least 12 months and preferably for 24 months.

In some additional embodiments, there is provided the above composition of this invention, wherein said composition is a ready to use product or is prepared in-situ by the patient before use from a concentrate or becomes an emulsion upon contact with a mammal's digestive tract fluids.

According to some embodiments, there is provided the above composition of this invention, wherein comprising from about 0.005% w/w to about 3.0% w/w of the at least one terpene or essential oil.

According to some other embodiments, there is provided the above composition of this invention, wherein the ratio between the at least one cannabinoid and the at least one terpene is from at least about 40:1 to about 1:1, and more preferably from about 30:1 to about 1:1 or from about 20:1 to about 1:1 or from about 10:1 to about 1:1 on weight basis.

By some embodiments, there is provided the above composition of this invention, wherein comprising from about 1% to about 10% by weight of at least one non-ionic hydrophilic emulsifier or a mixture of emulsifiers with a HLB value greater than 10, more preferably a HLB value of from about 10 to about 16.

In some other embodiments, there is provided the above composition of this invention, wherein one of the at least two emulsifiers is selected from non-ionic emulsifiers with a HLB value of about 10 to about 16 and a second emulsifier is selected from non-ionic hydrophilic or hydrophobic emulsifiers having a HLB value of about 4 to about 12.

In some additional embodiments, there is provided the above composition of this invention, wherein at least one of the at least two emulsifiers having a HLB value of from about 10 to about 16 is selected from tocopheryl polyethylene glycol 1000 succinate (TPGS), sucrose ester, polyethylene glycol castor oil, polysorbate, polyglyceryl fatty acid ester, and combinations thereof.

According to some embodiments, there is provided the above composition of this invention, wherein the at least two hydrophilic emulsifiers are selected from the group consisting of a polysorbate, polysorbate 80, oleoyl polyoxyl glycerides, polyoxyl hydrogenated castor oil, sucrose distearate, tocopherol polyethylene glycol 1000 succinate, lauroyl polyoxyl glycerides, a sorbitan fatty acid ester, sorbitan monooleate, polyglyceryl fatty acid ester, and polymeric emulsifier Acrylates/C10-30 Alkyl Acrylate Cross-Polymer, salts thereof, derivatives thereof and combinations thereof.

According to some other embodiments, there is provided the above composition of this invention, wherein the ratio between the at least one cannabinoid and the at least one emulsifier is from about 1:5 to 10:1, preferably from about 1:2 to about 5:1, and more preferably from about 1:1 to about 3:1.

In some additional embodiments, there is provided the above composition of this invention, wherein further comprising from about 0.1% w/w to about 5% w/w of at least one stabilizing or suspending agent.

According to some embodiments, there is provided a dosage form comprising the composition of this invention, wherein said dosage form is an emulsion, a syrup, a liquid, a pudding, a gel, wherein administered with a spoon or by a dropper or by a volume measuring device or mixed with nutrients or foods.

According to some other embodiments, there is provided a process for producing a stable composition of this invention by: A) mixing the at least one cannabinoid with the at least one triglyceride oil, the at least two emulsifiers and optionally the at least one terpene and heating to about 75 C, B) vigorous mixing, C) separately, preparing the water phase by heating water, to about 75° C., and dissolving under vigorous mixing inactive ingredients selected from a flavor, a taste masking agent, a colorant, an anti-oxidant, a microbial preservative and combinations thereof, D) mixing the two phases heated at about 75° C. under high shear homogenization.

By some embodiments there is provided a method of treatment of anxiety, insomnia, or a geriatric syndrome by administration to a mammal subject in need thereof a therapeutically effective amount of the composition or dosage form of this invention.

By some other embodiments there is provided a method of treating cannabinoid treatable disorders, by administering to a mammal subject in need thereof a therapeutically effective amount of the composition of this invention, wherein the composition exhibits an improved oral cannabinoid bioavailability, higher by at least about 100% than the bioavailability of the same at least one cannabinoid when dissolved in an organic solvent or mixture of organic solvents.

In some embodiments there is provided a method of treatment for inducing an alerting response by administration to a mammal subject in need thereof of a therapeutically effective amount of the composition of this invention, wherein said composition comprising at least one cannabinoid selected from at least about 1 mg of cannabidiol, a derivative thereof and combinations thereof and at least about 1 mg of at least one alerting terpene per one serving.

In some additional embodiments there is provided a method of treatment for inducing an alerting response, wherein the at least one alerting terpene is selected from limonene, alfa and beta pinene, orange terpenes, thymol, isoborneol, and isoeugenol or citronella or orange essential oils or lavender essential oil, caryophyllene, rosmarinus oil, citrus oil and combinations thereof.

According to some embodiments there is provided a method of treatment for sedation and/or sleep inducing in a mammal in need thereof, by administration at about one hour before retiring to sleep or as needed of a therapeutically effective amount of the composition of this invention, wherein comprising from about 1 mg to about 10 mg of at least one sedating cannabinoid and from about 0.5 mg to about 5 mg of at least one sedating terpene per unit serving.

According to some other embodiments, there is provided the above method for sedation and/or sleep inducing in a mammal in need thereof, wherein the sedating cannabinoid is selected from Tetrahydrocannabinol (THC), Cannabinol (CBN) and combinations thereof, and the sedating terpene is selected from myrcene, linalool, linalyl acetate, alfa terpineol and citronellal, sandalwood, lavender, valerian, neroli essential oils and combinations thereof.

According to some embodiments, there is provided the above method of treatment, whereas the at least one sedating terpene is selected from myrcene, linalool, linalyl acetate, alfa terpineol, terpinolene and citronellal, sandalwood, lavender, valerian, neroli oils and combination thereof and the at least one sedating cannabinoid is selected from Tetrahydrocannabinol (THC) and Cannabinol (CBN) and combinations thereof.

In some embodiments, there is provided any of the above methods of treatment of this invention, wherein the therapeutically effective amount of the composition of this invention, administered to a mammal subject in need thereof is in an amount determined by a dose-response study as known in the art or from about 0.1 ml to about 50 mL, preferably from about 0.2 mL to about 20 mL and more preferably from about 0.5 mL to about 5 mL per serving, as per doctor's prescription.

In some other embodiments, there is provided a kit for the treatment of geriatric syndromes and improvement of elderly wellbeing comprising a day composition and a night composition as above disclosed in this invention, comprising:

(i) A night time dosage form comprising
a. at least about 1 mg to about 20 mg of cannabis extract or at least one cannabinoid or derivatives thereof and
b. at least about 1 mg of at least one sedative terpene.
(ii) A day time composition comprising
a. at least about 10 mg cannabis extract or at least one cannabinoid or derivatives thereof having a CBD:THC ratio of about 10 to about 200 and
b. at least about 1 mg of at least one alerting terpene.

In some additional embodiments, there is provided a kit for the treatment of neurological patients, epilepsy, PTSD, anxiety, schizophrenia and Parkinson disease that are also suffering from insomnia and improvement of their wellbeing comprising a day composition and a night composition of this invention, comprising:

A night time dosage form comprising
at least about 1 mg to about 20 mg of cannabis extract or at least one cannabinoid or derivatives thereof and
at least about 1 mg of at least one sedative terpene.
A day time composition comprising
at least about 10 mg cannabis extract or at least one cannabinoid or derivatives thereof having a CBD:THC ratio of about 10 to about 200 and
at least about 1 mg of at least one alerting terpene.

The inventor has unexpectedly discovered that it is possible to develop an emulsified cannabinoid composition, comprising the desired cannabinoids and optionally terpenes mixture in an effective dose, that has good shelf life stability and improved oral bioavailability. Such findings led to the discovery of the pharmaceutical compositions and formulations of the present invention, which are demonstrated herein to possess several therapeutically-beneficial properties. For example, the pharmaceutical, medicinal or veterinary compositions of the present invention comprise mixture of cannabinoids and terpenes that are tailored and assembled for specific pharmacological need. Secondly, the compositions of the present invention are stable emulsions providing long shelf life stability at ambient room temperature of at least one year and for two years. Furthermore, the pharmaceutical compositions of the present invention are emulsified cannabinoid composition having very fine submicron droplets size, thus improving the oral bioavailability of the cannabinoids by at least 100% and more preferably by 200%, 300% or more.

In certain embodiments, the pharmaceutical composition comprises about 1% to about 25% by weight of at least one triglyceride oil or a mixture of oils. In certain embodiments, the pharmaceutical composition comprises about 1% to about 10% by weight of an oil or a mixture of oils. In certain embodiments, the oil is selected from the group consisting of vegetable oils, such as; borage oil, coconut oil, cottonseed oil, soybean oil, safflower oil, sunflower oil, castor oil, corn oil, olive oil, palm oil, peanut oil, peppermint oil, poppy seed oil, canola oil, hydrogenated soybean oil. In a certain embodiment a prefers triglyceride oils is capric/caprylic triglycerides. In a preferred embodiment a preferred oil is olive oil.

In certain embodiments, the pharmaceutical composition of the emulsified cannabinoid has a plurality of particles having a mean particle size of 5 microns or less. In certain embodiments, the pharmaceutical composition of the emulsified cannabinoid has a plurality of particles having a mean particle size of 2 microns or less. In certain embodiments, the pharmaceutical composition of the emulsified cannabinoid has a plurality of particles having a mean particle size of 1 micron or less. In certain embodiments, the pharmaceutical composition of the emulsified cannabinoid has a plurality of particles having a mean particle size of 0.5 microns or less.

In certain embodiments, the emulsion cannabinoid composition further comprises a stabilizing agent which is a viscosity modifier and suspending agent, which is a high-molecular-weight polymer or a mixture of high-molecular-weight polymers. In certain embodiments, the high-molecular-weight polymer is selected from the group consisting of cellulose derivatives such as ethyl cellulose or hydroxypropyl methyl cellulose (HPMC), hydroxyl ethyl cellulose, cellulose phthalate, microcrystalline cellulose, polyvinylpyrrolidone (PVP), polyvinyl alcohol (PVA), polyvinyl caprolactam, polyvinyl acetate, polyethylene glycol graft copolymer, acrylates and acrylic polymers, methyl acrylate, methacrylic acid/ethyl acrylate copolymers, alkyl acrylate or cross linked acrylates, natural polysaccharides, such as xanthan gum, guar gum, locust bean gum, gum arabica, pectin, zein, karaya gum, alginate, hyaluronic acid, chitosan, starch, polyethylene glycols or polyethylene glycols and polypropylene glycols block copolymers, and mixtures thereof.

In certain embodiments, the high-molecular-weight polymer is selected from the group consisting of microcrystalline cellulose (MCC) (Avicel™), cellulose derivatives, acrylate and alkyl acrylate cross polymer (Pemulene™), xanthan gum (Xantural™), acacia, tragacanth, sodium carboxy methyl cellulose and MCC: alginate complex compositions.

In certain embodiments, the emulsion cannabis composition is stable towards freezing. In certain embodiments, the emulsion cannabis composition comprises a sugar, a poly sugar or a polymer that are cryoprotectants, such as sucrose, lactose, mannitol, sucrose syrup USP, polymers such as polyvinylpyrrolidone, alkyl acrylate cross copolymer, acrylate polymer, polysaccharide, as examples. In certain embodiments, the emulsion cannabinoid composition does not cream or separate upon a freeze and thaw cycle.

In certain embodiments, the dosage form comprising the composition of this invention is formulated as a liquid, a syrup or enema. In certain embodiments, the dosage form is formulated for oral or mucosal delivery. In certain embodiments, the dosage form is formulated as or in a lozenge, candy, toffee, chocolate or cookie.

In certain embodiments, any one of the compositions described above, or any one of the dosage forms described above, is for use in a method of treating a cannabinoid-responsive symptom, disease or disorder.

In certain embodiments, the composition or dosage form comprises cannabidiol (CBD). In certain embodiments, the pharmaceutical composition or dosage form further comprises tetrahydrocannabinol (THC). In certain embodiments, the CBD:THC weight ratio is about 20:1. In certain embodiments, the mixture of a cannabinoid and a terpene is a cannabinoid extract. In certain embodiments, the mixture of a cannabinoid and a terpene is a cannabis extract. In certain embodiments, the mixture comprises CBD. In certain embodiments, the mixture comprises THC. In certain embodiments, the mixture comprises CBD and THC. In certain embodiments, the mixture comprises CBD and THC in a weight ratio of about 1:1. In certain embodiments, the mixture comprises CBD and THC in a weight ratio of about 10:1 to about 1:10.

In certain embodiments, the oil phase of the oil-in-water pharmaceutical composition further comprises about 0% to about 30% by weight of tats or waxes, such as fatty acid or fatty alcohol, glyceryl or propylene glycol mono or di stearate, fats, lipids, oils other than essential oils, co-solvents or mixtures thereof, whereas the fatty acids are liquid or solid at room temperature.

In certain embodiments, the external aqueous phase of the oil-in-water cannabinoid emulsion is sucrose syrup such as USP sucrose syrup, or honey, or flavored syrup such as raspberry or mint or peppermint flavored syrup, or chocolate syrup, or maple syrup, or dates honey (Silane), tamarind honey, and the like known in the art.

In certain embodiments the composition is a ready to use emulsion, of a sub-micron type or of a nano-size type. In certain embodiments the emulsified composition is an essentially waterless concentrate ready to be diluted with aqueous medium before use or to be diluted upon contact with a mammal's subject body fluids, such as saliva, gastric or intestinal fluids.

The method of production of the emulsion or emulsified composition comprises applying heat and homogenization. Homogenization is carried out preferably by high shear in line homogenizer or high-pressure homogenizer. Further details of suitable methods for producing emulsions and dosage forms may be obtained from any standard reference work in this field, including, for example: Remington's Pharmaceutical Sciences, Mack Publishing Co, Easton, Pa., USA (1980).

In certain embodiments, the production process of the emulsified cannabinoid composition is performed by stator and rotor type homogenizer that are simple to scale up, such as high shear homogenizer, for example Silverson™ or Magiclab™ and there is no need for sophisticated and more expensive production methods and equipment such as high pressure homogenization, for example Microfluidizer™.

In certain embodiments, the production process of the cannabinoid emulsion is performed by heating separately the oily phase, comprising the cannabinoid, the oil, the emulsifiers and oily additives and the water phase, and combining the two phases under moderate agitation and fast cooling to room temperature.

### Definitions

The term “pharmaceutical composition” as used herein has its conventional meaning and refers to a composition which is pharmaceutically acceptable. The term “pharmaceutically acceptable” as used herein has its conventional meaning and refers to compounds, materials, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The term “excipient” as used herein has its conventional meaning and refers to a pharmaceutically acceptable ingredient, which is commonly used in the pharmaceutical technology for preparing a pharmaceutical composition, such as a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes. The term “veterinary composition” encompasses the full range of compositions for internal administration and feeds and drinks which can be consumed by animals.

The term “essentially free” of micelles or liposomes means that at least 90% and preferably, 95% or 98% of the cannabinoids and the triglyceride oil are in the form of emulsion oil droplets comprising the internal phase of the emulsions, and the amount of residual cannabinoids and the triglyceride oils are solubilized in the forms of micelles or liposomes, comprises less than 10% on weight basis of the emulsion composition and preferably less than 5% or 2%.

The term “stable emulsion” means an emulsion that does not separate or cream or shows any other significant visual change within about 12 months at ambient room temperature and more preferable over 18 months at ambient temperature and more preferable 24 months at ambient room temperature.

The term “oral bioavailability” in this context means the amount of the specific cannabinoid measured in blood of mammals over time, and is calculated as the area under the curve (AUC) of the blood levels over time to end of study or end of 24 hours or infinite time.

The term “cannabinoid” as used herein generally refers to one of a class of diverse chemical compounds that act on a cannabinoid receptors.

The term “cannabis extract” as used herein refers to one or more plant extracts from the cannabis plant. A cannabis extract contains, in addition to one or more cannabinoids, one or more non-cannabinoid components which are co-extracted with the cannabinoids from the plant material. Their respective ranges in weight will vary according to the starting plant material and the extraction methodology used. Cannabinoid-containing plant extracts may be obtained by various means of extraction of cannabis plant material. Such means include but are not limited to: supercritical or subcritical extraction with CO₂, extraction with hot or cold gas and extraction with solvents.

The term “essential oil” or “essential oils” as used herein relates to a concentrated hydrophobic liquid oil containing volatile aroma compounds, obtained from plants. Essential oils are also known as volatile oils, ethereal oils, due their distinct typical strong volatility at ambient temperature, or simply as the oil of the plant from which they were extracted, such as “oil of lavender”. An oil is “essential” in the sense that it contains the “essence of” the plant's fragrance—the characteristic fragrance of the plant from which it is derived.

Essential oils are generally extracted by distillation, often by using steam. Other processes include expression, solvent extraction, absolute oil extraction, resin tapping, and cold pressing. They are used in perfumes, cosmetics, soaps and other products, for flavoring food and drink, and for adding scents to incense and household cleaning products. The essential oils are comprised mainly of terpenes or terpenoids and the various properties of the different essential oils are derived from their terpenes content.

The term “terpene” as used herein also includes terpenoids. Terpenes are lipophilic compounds, volatile and liquid at room temperature and are used herein in this invention as part of the composition that contribute to increased bioavailability as well as cannabinoids solubilizers and as pharmacologically active agents that works in synergy or entourage with the cannabinoids. Terpenes are volatile organic compounds formed by the union of hydrocarbon of 5 carbon atoms, known as isoprene. The smallest and most volatile compounds are monoterpenes, which are biosynthesized by the union of two isoprene molecules. The biggest and least volatile are biosynthesized by the union of three or more isoprene molecules. The sesquiterpenes are next in the chain, which are formed by the union of three isoprene molecules. Terpenes are secondary metabolites, which provide the plant with its organoleptic characteristics (aroma and flavor) and that constitutes most of the essential oil produced by aromatic plants. Terpenes are major secondary metabolites of cannabis and are responsible for the odor and flavor of various cannabis strains. Cannabis strains and hemp strains produce many terpenes as secondary metabolites. Terpenes are synthetized from terpene unit into mono-terpenes, sesqui-terpenes, di-terpenes that are lipophilic, volatile and insoluble in water and are cyclic or bicyclic or not cyclic and may have alcohol, aldehyde or ketone chemical moiety. The term “terpene” further relates to essential oils. The term “terpene” does not include fats and/or lipids.

The term “about” as used herein refers to any value which lies within a range of ±5% of original value. For example, “about 100” refers to “95 to 105”.

The term “triglyceride oil” is used here is an oil made of three fatty acids conjugated by an esters bond to glycerin or glycerol, whereas the fatty acids are having from C8 C22 carbons. The fatty acids may be unsaturated or saturated and hydrogenated or not. The triglyceride oil may also comprise diglycerides and free fatty acids in small amount. The triglyceride can be liquid or solid at room temperature. A preferred type of triglyceride is capric/caprylic triglyceride that is less prone to oxidation.

The term “emulsifier” as used herein is an amphiphilic molecule that is surface active agents which stabilizes emulsions by reducing the interfacial tension.

As used herein, the term “particles” as used herein relates to droplets. The term “particle size” of an emulsion is to be understood also as the “droplet size” of that emulsion. The term “mean particle size” is also to be understood as the term “mean droplet size”.

As used herein, the term “mean particle size” refers to a value which is obtained by measuring the diameters in a specific direction of particles and dividing the sum of respective diameters of particles by the number of measured particles.

The methods, uses, materials, and examples that will now be described are illustrative only and are not intended to be limiting; materials, uses and methods similar or equivalent to those described herein can be used in practice or testing of the invention. Other features and advantages of the invention will be apparent from the following figures, detailed description, and from the claims.

### BRIEF DESCRIPTION OF THE DRAWINGS

FIG. 1 DLS particle size results of example 1A table 2.

FIG. 2 DLS particle size results of example 517 table 11.

FIG. 3 Light microscope picture X200 517 table 11.

### DETAILED DESCRIPTION OF THE INVENTION

Provided by the present invention are cannabinoid syrup compositions comprising at least one cannabinoid, solubilized in the internal oil phase of an oil-in-water type sub-micron emulsion, having increased oral bioavailability in comparison to the same cannabinoid dissolved in organic solvent or mixture of organic solvents, and over one-year stability at ambient room temperature and comprises taste-enhancing excipients to reduce the bitterness of cannabinoids.

Without being limited to any theory or mechanism, the present invention is based on the surprising finding that a composition of

a: at least one cannabinoid
b: optionally at least one terpene,
c: at least one vegetable oil,
d: at least two emulsifiers in specific concentrations,
when emulsified in aqueous medium, is exceptionally proficient in producing stable oil-in-water type submicron or nano-emulsions and stable cannabis products which are patient-friendly and exhibit high oral bioavailability and devoid of the unpleasant bitter cannabis or cannabinoids taste.

The inventor of this invention has unexpectedly discovered that the emulsified compositions comprising at least one cannabinoid of this invention exhibit improved oral bioavailability of at least about 100% higher and in a preferred embodiment of about 100% to 200%, from about 200% to about 300% higher and higher than 300% as compared to the oral bioavailability of the same cannabinoid when dissolved in an organic solvent or mixture or organic solvents of ethanol and propylene glycol.

It has been unexpectedly discovered that during the preparation of the emulsions of this invention, stable sub-micron or nano-emulsions are produced only when the oily internal phase of the oil-in-water emulsion is below about 10% of the total weight, and preferably below 8%, on a weight basis, and more preferably below 6%, and the emulsions produced under these conditions are stable compositions that enable long shelf life of about two years and improved oral bioavailability of the cannabinoids in mammals.

It has been unexpectedly discovered that robust or rugged nano-emulsions or sub-micron emulsions of the oil-in-water type comprising cannabinoids are produced only in a specific set of conditions and compositions, wherein the at least two emulsifiers are selected from a group of emulsifiers, one having an HLB value of from about 10 to about 16 and the second having HLB value of from about 2 to about 8 and preferably at least one of the emulsifiers is selected from sucrose fatty acid esters; such as sucrose stearate, polyglyceryl fatty acid esters, polyoxyl castor oil, polysorbates and tocopheryl PEG1000 succinate, possessing the much-desired long shelf life stability and improved oral bioavailability.

The present invention provides, in one aspect, an emulsified composition comprising from about 0.02% w/w to about 10% w/w of at least one cannabinoid or a mixture of cannabinoids, from about 1% w/w to about 10% w/w of a triglyceride oil, and from about 1% w/w to about 10% w/w of at least two emulsifiers, wherein upon homogenizing in the water external phase of the emulsion or upon mixing with mammals body fluids, the composition obtained exhibits a plurality of particles having a mean particle size of from about 10 nm to about 10 m and more preferably from about 20 nm to about 2,000 nm, or from about 50 nm to about 1,000 nm, or from 80 nm to 800 nm, or from 90 nm to about 600 nm, or from about 100 nm to about 400 nm, or from 10 nm to about 200 nm.

The present invention provides, in one embodiment, an emulsion composition, comprising from about 0.02% w/w to about 10% w/w of at least one cannabinoid, from about 1% w/w to about 10% w/w of a triglyceride oil, and from about 1% w/w to about 10% w/w of at least two emulsifiers, wherein the ratio of the at least one triglyceride oil to at least two emulsifiers is about 1:10 to about 10:1 and more preferably from about 1:1 to about 4:1, and wherein the emulsion composition has a plurality of particles having a mean particle size of from about 10 nm to about 2,000 nm, more preferably from 10 nm to 1,000 nm and more preferable from about 10 nm to 800 nm or from 100 nm to 600 nm.

The emulsion composition of this invention is a very fine opaque to translucent emulsion. The sub-micron or nano-size range is from about 10 nm to about 1,000 nm and from about 10 nm to about 800 nm and more particularly from about 10 nm to about 600 nm, or from about 10 nm to about 400 nm, or from about 10 nm to about 300 nm, or from about 10 nm to about 200 nm.

In certain embodiments, the “emulsified composition” may be produced as known in the art of emulsion manufacturing, by heating the oil phase and the water phase separately, and mixing them under high shear homogenization process, or producing the oily phase by heating and agitation to produce a composition that forms an emulsion upon mixing with an aqueous medium, such as the external aqueous phase of the emulsion or mammals body fluids, by process of self-emulsification or by applying vigorous mixing such as high shear homogenization.

In certain embodiments, the pharmaceutical composition of the present invention is stabilized with a mixture of emulsifiers, to produce a composition that is stable at ambient room temperature at least 12 months or about 18 months or about 24 months. Stability is evident by stable “content uniformity” tested as the amount of the cannabinoids in different unit doses, or according to USP procedure, and physical stability of the emulsion.

In certain embodiments, the pharmaceutical composition of the present invention is a non-liposomal composition.

In certain embodiments, the pharmaceutical composition of the present invention is a non-micellar composition.

In certain embodiments, the pharmaceutical composition of the present invention is a non-liposomal and non-micellar composition.

In certain embodiments, the particle or particles of the present invention are non-liposomal particle or particles. In certain embodiments, the particle or particles of the present invention are non-micellar particle or particles. In certain embodiments, the particle or particles of the present invention are non-liposomal and non-micellar particle or particles. Each possibility represents a separate embodiment of the invention. The terms “non-liposomal” and “non-micellar” as used herein refer to compositions and particles devoid or substantially devoid of liposomes and/or micelles.

In certain embodiments, a centrifugation test is applied in order to estimate separation phenomenon and occurrence of separation or creaming over time in an accelerated manner. Emulsions that do not exhibit creaming, separation or segregation by visual inspection following centrifugation at about 3,500 RPM for about five minutes are predicted to have a shelf life of at least 12 months and about 24 months and are not influenced by gravitation as per physical stability and separation phenomenon.

In certain embodiments, the emulsified composition is an emulsion, wherein said emulsion is stable for at least 12 months. In certain embodiments the emulsified composition is a stable emulsion at ambient temperature with a shelf life of over 18 months or over 24 months. In certain embodiments, the emulsion is of oil-in-water type, and the external aqueous medium comprises essentially water- and water-soluble ingredients. In certain embodiments, the aqueous medium comprises stabilizing agents, additives such as colorants, sweeteners, taste masking agents, taste-enhancing excipients, flavors, anti-oxidants, pH adjusting agents such as buffers and microbial preservatives.

A taste-enhancing excipients are sweeteners and taste masking agent that may be any sugar, natural or artificial sweetener, for example sucrose, glucose, sucralose, glycerin, aspartame, cyclamate, stevia, erythritol, xylitol, acesulfam, honey, sugar syrup, maple syrup, dates syrup (silane), tamarhind syrup, raspberry syrup, glycyrrhizin, glycyrrhitinic acid, Glycyrrhiza glabra extracts and combinations, and flavors, as for example, orange and mint, glycyrrhizin, glycyrrhitinic acid, Glycyrrhiza glabra extracts, such as Magnasweet™ (Monoammonium Glycyrrhizinate, dipotassium glycyrrhizinate, di and trisodium glycyrrhizinate, stearyl glycyrrhizinate, ammoniated glychyrhhizin), provided by Mafco Corp, products and used in the food and pharma industry to mask unwanted tastes, or glycerin, to mask the unpleasant taste of low to moderately bitter or earthy taste of the cannabis extracts. In addition, effervescent agents (sodium bicarbonate, citric acid) can also be added to improve the mouth feel. Some formulations may include a bitterness blocking agent that masks the bitter taste or the perception of bitter on the tongue. Such bitter blockers may include adenosine monophosphate, lipoproteins, or phospholipids. These agents compete with the bitter active to bind to the G-protein coupled receptors on the tongue (receptor sites that detect bitter), thus suppressing the bitter taste. It has also been found that sodium chloride can be added to a formulation to mask bitterness as in the preparation of pioglitazone hydrochloride orally disintegrating tablets.

In certain embodiment taste enhancing excipient are sucralose which is an artificial sweetener 300 time sweeter than sucrose, and monoammonium glycyrrhizinate which is eliminating aftertastes, intensifying sweetness, extending sweetness and enhancing other flavors.

In a certain embodiment a mixture of taste enhancing excipients are used for effective reduction of the bitter taste of cannabinoids, for example taste-enhancing excipient is comprised of about 2% w/w of sucralose, about 0.5% w/w of monoammonium glycyrrhizinate (Magnasweet™ 110), and a syrup elected from sucrose syrup, maple syrup, raspberry syrup or artificial syrup make up to 100% by weight.

In certain embodiments the emulsion composition of this invention is stable at regular or accelerated temperature conditions, wherein the emulsion does not separate or cream and mean particle size does not change more than about 20% while stored three month at 40° C., or about 12 month or about 18 months or about 24 months at ambient temperature and the content uniformity of the cannabinoid and properties such as viscosity, pH, Zeta potential, color, taste and osmolarity do not change more than about 20% and preferably less than about 10% of the value tested at zero time.

In certain embodiments, the pharmaceutical composition further comprises about 0.5% to about 5%, or about 0.2% to about 2% by weight of at least one stabilizing or viscosity-modifying or suspending agent or a mixture of stabilizing or viscosity modifying or suspending agents.

The term “viscosity modifier” or “a viscosity-modifying agent” as used herein refers to any additive that can modify the pharmaceutical composition viscosity to a desired viscosity level (either higher or lower). Typical example includes, without limitation solvents, lubricants and gelling agents, such as carboxy methylcellulose, the variety of modified cellulose such as hydroxy methyl cellulose, poly vinyl pyrrolidine, acrylates and the same. The viscosity modifier component can comprise one or more viscosity modifier. As used herein, the term “viscosity modifier” is further intended to mean a compound or mixture of compounds that can be used to adjust the viscosity of a composition of the invention. Suitable viscosity modifiers include microcrystalline wax, glyceryl dibehenate, hydrogenated castor oil wax MP80, and others recognized by artisans in the field. Suitable viscosity may be obtained by use of dense syrups such as sucrose syrup USP as the external water phase of the oil in water type emulsion. In certain embodiments, the viscosity modifier is selected from the group consisting of microcrystalline wax, glyceryl dibehenate, hydrogenated castor oil wax MP80, and any combination thereof. Each possibility represents a separate embodiment of the invention.

In certain embodiments, the pharmaceutical composition comprises from about 0.01% w/w to about 20% w/w of at least one cannabinoid. In certain embodiments, the pharmaceutical composition comprises from about 0.02% w/w to about 10% w/w of at least one cannabinoid. In certain embodiments, the pharmaceutical composition comprises from about 0.05% w/w to about 5% w/w of at least one cannabinoid. In certain embodiments, the pharmaceutical composition comprises from about 0.1% w/w to about 2% w/w of at least one cannabinoid.

In certain embodiments, the cannabinoid is a natural cannabinoid. In certain embodiments, the cannabinoid is a natural cannabinoid found in a Cannabis plant. In certain embodiments, the cannabinoid is a synthetic cannabinoid. In certain embodiments, the cannabinoid is a mixture of natural cannabinoids. In certain embodiments, the cannabinoid is a mixture of synthetic cannabinoids. In certain embodiments, the cannabinoid is a mixture of natural and synthetic cannabinoids.

The term “natural cannabinoid” as used herein generally refers to a cannabinoid which can be found in, isolated from and/or extracted from a natural resource, such as plants. “Synthetic cannabinoids” are a class of chemicals that are different from the cannabinoids found e.g. in cannabis but which also bind to cannabinoid receptors.

In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabinol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

The terms “cannabidiol” and “CBD” are interchangeably used herein and refer to a non-psychotropic cannabinoid having the structure depicted in Formula I below and salts or derivatives thereof, such as Δ4-cannabidiol, Δ5-cannabidiol, Δ6-cannabidiol, Δ1,7-cannabidiol, Δ1-cannabidiol, Δ2-cannabidiol, Δ3-cannabidiol.

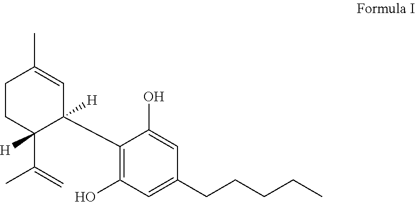 [SMILES: C=C(C)[C@@H]1CCC(C)=C[C@H]1c1c(O)cc(CCCCC)cc1O]

In some other embodiments, the pharmacologically active cannabinoid is selected from the group consisting of tetrahydrocannabinol, Δ9-tetrahydrocannabinol (THC), Δ8-tetrahydrocannabinol, standardized marijuana extracts, Δ8-tetrahydrocannabinol-DMH, Δ9-tetrahydrocannabinol propyl analogue (THCV), 11-hydroxy-tetrahydrocannabinol, 11-nor-9-carboxy-tetrahydrocannabinol, 5′-azido-.Δ8-tetrahydrocannabinol, AMG-1 (CAS Number 205746-46-9), AMG-3 (CAS Number 205746-46-9), AM-411 (CAS Number 212835-02-4), (-)-11-hydroxy-7′-isothiocyanato-Δ8-THC (AM-708), (-)-11-hydroxy-7′-azido-Δ8-THC (AM-836), AM-855 (CAS Number 249888-50-4), AM-919 (CAS Number 164228-46-0), AM926, AM-938 (CAS Number 303113-08-8), cannabidiol (CBD), cannabidiol propyl analogue (CBDV), cannabinol (CBN), cannabichromene, cannabichromene propyl analogue, cannabigerol, CP 47,497 (CAS Number (1S,3R): 114753-51-4), CP 55,940 (CAS Number 83002-04-4), CP 55,244 (CAS Number 79678-32-3), CT-3 (ajulemic acid), dimethylheptyl HHC, HU-210 (1,1-Dimethylheptyl-11-hydroxy-tetrahydrocannabinol), HU-211 (CAS Number 112924-45-5), HU-308 (CAS Number 1220887-84-2), WIN 55212-2 (CAS Number 131543-22-1), desacetyl-L-nantradol, dexanabinol, JWH-051 (Formula C25H3802), levonantradol, L-759633 (Formula C26H4002), nabilone, 0-1184, and mixtures thereof.

In other embodiments, the pharmacologically active cannabinoid may further be selected from the group consisting of palmitoylethanolamide (PEA), alkylethanolamide, oleyl-serine, a cannabinomimetic, caryophyllene, CB1 and/or CB2 agonist and/or antagonist, partial agonist, reversible or not, and combinations thereof.

The cannabinoid may be included in the composition of this invention in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; and enol forms.

In some embodiments, the at least one cannabinoid used in the present invention is a lipophilic concentrate or isolate of cannabinoid(s). In some embodiments, the cannabinoid(s) used in the present invention are a lipophilic concentrate of cannabinoid(s) achieved via CO2, solvents or gas extraction techniques, by oil maceration or oil pressure of plant parts or of the whole plant.

Extraction of cannabis plant of various plant parts, may be done by CO2 extraction or by solvent extraction or solvent-less compression to obtain oily viscous material or waxy material or solid material, depending on plant material, plant parts and extraction methods as skilled in the art. Extraction and processing may result in a broad spectrum of cannabis molecules, cannabinoids, terpenes and other families of natural cannabis molecules or in a pure extract of cannabinoids or concentrated cannabinoid terpenes extract. Cannabis or marijuana extract may be further decarboxylated, winterized and/or purified, for example by distillation, as known in the art.

In certain embodiments, the composition of this invention comprises from about 0.01% w/w to about 5% w/w of at least one terpene or essential oil and mixtures thereof. In certain embodiments, the composition comprises from about 0.05% w/w to about 4% w/w of at least one terpene or mixtures thereof. In certain embodiments, the pharmaceutical composition comprises from about 0.05% w/w to about 1% w/w of a terpene or a mixture thereof. In certain embodiments, the pharmaceutical composition comprises about 0.5% w/w to about 4% w/w of at least one terpene or mixtures thereof. In certain embodiments, the pharmaceutical composition comprises from about 0.5% w/w to about 3% w/w of at least one terpene or mixtures thereof. In certain embodiments, the pharmaceutical composition comprises from about 1% w/w to about 2% w/w of at least one terpene or mixtures thereof.

In certain embodiments, the pharmaceutical composition comprises from about 0.01% w/w to about 20% w/w of a mixture of at least one cannabinoid and at least one terpene or essential oil. In certain embodiments, the pharmaceutical composition comprises from about 0.02% w/w to about 10% w/w of a mixture of at least one cannabinoid and at least one terpene. In certain embodiments, the pharmaceutical composition comprises from about 0.05% w/w to about 5% w/w of at least one cannabinoid and at least one terpene. In certain embodiments, the pharmaceutical composition comprises from about 0.5% w/w to about 2% w/w of a mixture of at least one cannabinoid and at least one terpene. In certain embodiments, the pharmaceutical composition comprises from about 0.001% w/w to about 1% w/w of a mixture of at least one cannabinoid and at least one terpene. In certain embodiments, the pharmaceutical composition comprises from about 0.05% w/w to about 0.5% w/w of at least one cannabinoid and at least one terpene.

In certain embodiments of the pharmaceutical composition, the weight ratio between the cannabinoid and the terpene is from about 25:1 to about 1:1. In certain embodiments of the pharmaceutical composition, the weight ratio between the cannabinoid and the terpene is from about 20:1 to about 1:1. In certain embodiments of the pharmaceutical composition, the weight ratio between the cannabinoid and the terpene is from about 15:1 to about 1:1. In certain embodiments of the pharmaceutical composition, the weight ratio between the cannabinoid and the terpene is from about 10:1 to about 1:1. In certain embodiments of the pharmaceutical composition, the weight ratio between the cannabinoid and the terpene is from about 5:1 to about 1:1.

In certain embodiment, there is provided a high strength cannabinoid syrup, comprising at least 200 mg of cannabinoids in a single teaspoon, or at least 400 mg of cannabinoid per one teaspoon or at least 500 mg of cannabinoid in one teaspoon.

In certain embodiments, the terpene is a natural terpene. In certain embodiments, the terpene is a natural terpene found in a Cannabis plant. In certain embodiments, the terpene is a synthetic terpene. In certain embodiments, the terpene is a mixture of natural terpenes. In certain embodiments, the terpene is a mixture of synthetic terpenes. In certain embodiments, the terpene is a mixture of natural and synthetic terpenes.

In certain embodiments, the terpene is selected from the group consisting of bisabolol, borneol, caryophyllene, carene, camphene, cineol, citronella, eucalyptol, geraniol, guaiol, humulene, isopropyltoluene, isopulegol, linalool, limonene, methyl salicylate, menthol, myrcene, nerolidol, ocimene, pinene, phytol, pulegone, terpinene, terpinolene, thymol, salts thereof, derivatives thereof and mixtures thereof. Each possibility represents a separate embodiment of the invention.

In certain embodiment the terpene is a cannabis plant terpene, or a terpene derived from a non-cannabis plant material or a synthetic terpene. In certain embodiment the terpene is a taste modifier or smell modifier agent, a food grade or pharmaceutical grade, a solubilizer or solvent and an excipient in the formulation.

120 distinct terpenes are produced by the genus Cannabis, with the relative concentrations of the individual terpenes varying greatly among the 700 distinct strains currently in cultivation. Aside from taste and smell differences between varieties, this helps contribute to the broad diversity of potential medical applications of Cannabis.

In certain embodiments of the pharmaceutical composition, the natural cannabinoid is derived or isolated from an extract of a Cannabis plant. In certain embodiments of the composition, the natural terpene is derived or isolated from an extract of a Cannabis plant.

In certain embodiments the weight ratio in the composition of this invention, between the at least one cannabinoid and the at least two emulsifiers is from about 1:5 to about 10:1. In certain embodiments, the weight ratio between the at least one cannabinoid and the at least two emulsifiers is about 1:2 to about 5:1. In certain embodiments, the weight ratio between the at least one cannabinoid and the at least two emulsifiers is about 1:2 to about 4:1. In certain embodiments, the weight ratio between the at least one cannabinoid and the at least two emulsifiers is about 1:2 to about 3:1. In certain embodiments, the weight ratio between the at least one cannabinoid and the at least two emulsifiers is about 1:2 to about 2:1.

The emulsifier ingredients of the composition of this invention improve the cannabinoid solubilization and the emulsifying properties of the formulation. The emulsifiers, also termed surfactants, are selected from the group consisting of polyglycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glycerol esters of saturated C8-C18 fatty acids (Gelucire® 33/01), glyceryl esters of saturated C12-C18 fatty acids (Gelucire® 39/01 and 43/01), glyceryl laurate/PEG-32 laurate (Gelucire® 44/14), glyceryl laurate glyceryl/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), lecithin, phospholipids and mixtures thereof. Polyglycolized derivatives and polyoxyethylene esters or ethers derivatives of medium to long chain fatty acids, commercially named Brij and Myrj variety surfactants, and propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, glyceryl caprylate/caprate, medium chain (C8/C10) mono- and diglycerides (Capmul® MCM, Capmul® MCM (L)), mono- and diacetylated monoglycerides, polyglyceryl oleate, polyglyceryl-2 dioleate, polyglyceryl-3 dioleate, polyglyceryl-10 trioleate, polyglyceryl-10 laurate, polyglyceryl-10 oleate, and polyglyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, and mixtures thereof. Sucrose esters surfactants such as sucrose stearate, sucrose distearate, sucrose palmitate, sucrose oleate and polyethylene glycol sorbitan fatty acid esters, which can be used, include PEG-20 sorbitan monolaurate, PEG-20 sorbitan monopalmitate, PEG-20 sorbitan monostearate, and PEG-20 sorbitan monooleate, and TPGS (d-.alpha.-tocopheryl polyethylene glycol 1000 succinate), polysorbate 20 (Tween® 20), polysorbate 80 (Tween® 80), polyethyleneglycol 660 12-hydroxystearate (Solutol® HS-15 or Kolliphor® HS15), polyglyceryl-3 dioleate (Plurol® Oleique CC 497, Gattfosse), Acrylates/C10-30 Alkyl Acrylate Cross-Polymer (Pemulene™ TR2, Lubrizol). sodium lauryl sulfate and combinations thereof.

Polyoxyethylene-polyoxypropylene block copolymers, which can be used as emulsifiers in the compositions of this invention include poloxamers (108, 124, 182, 183, 188, 212, 217, 238, 288, 331, 338, 335, and 407), and mixtures thereof. Sorbitan fatty acid esters, which can be used, include sorbitan monolaurate (Span® 80), sorbitan monopalmitate, sorbitan monooleate (Span® 20), sorbitan monostearate, sorbitan tristearate and combinations thereof.

A preferred type of emulsifiers is polymeric surfactant, such as Acrylates/C10-30 Alkyl Acrylate Cross-Polymer. Or alkyl acrylate cross-polymer, Pemulen™ TR1 or TR2, from Lubrizol Ltd.

In certain embodiments, the composition comprises from about 0.5% w/w to about 15% w/w of at least two emulsifiers. In certain embodiments, the pharmaceutical composition comprises from about 1% w/w to about 5% w/w of at least two emulsifiers. In certain embodiments, the two emulsifiers are selected from the group consisting of polysorbate 80, polyoxyl 35 hydrogenated castor oil, sucrose fatty acids esters such as, sucrose stearate and sucrose distearate, tocopherol polyethylene glycol 1000 succinate, polyglyceryl-3 dioleate, sorbitan monooleate, sorbitan monooleate, sucrose tatty acid ester, alkyl acrylate cross-polymer and salts thereof, derivatives thereof and combinations thereof.

In certain embodiments, a mixture of at least two emulsifiers is more effective in providing stable and low particle size of emulsified cannabinoid composition. In a preferred embodiment, at least one of the emulsifiers is a sucrose fatty acid ester selected from sucrose stearate, sucrose distearate, sucrose oleate, sucrose palmitate, sucrose laurate, sucrose tetrastearate and sucrose polystearate. Preferred sucrose esters are sucrose stearates for example Surfhope™ D-1811F, Surfhope™ D-1815, Surfhope™ D-1615 all from Mitsubishi chemicals, Crodesta™ F110 which is a mixture of mono and diesters with a HLB value of 12, or Crodesta™ F160 which is a monoester with a HLB value of 14.5.

In certain embodiments the HLB of the at least one emulsifier is about HLB 8 to about HLB 18, In certain embodiments the HLB of the at least one emulsifier is about HLB 8 to about HLB 16, In certain embodiments the HLB of the at least one emulsifier is about HLB 10 to about HLB 16, In certain embodiments the HLB of the at least one emulsifier is about HLB 8 to about HLB 15, In certain embodiments the HLB of the at least one emulsifier is about HLB 11 to about HLB 16, In certain embodiments the HLB of the at least one emulsifier is about HLB 11 to about HLB 15, In certain embodiments the HLB of the at least one emulsifier is about HLB 11 to about HLB 14. Each possibility represents a separate embodiment of the invention.

In some preferred embodiments the average HLB value calculated on the molar basis HLB of the mixture of the emulsifiers is from about HLB 6 to about HLB 16. In certain embodiments the combined HLB calculated on the molar basis HLB of the mixture of the emulsifiers is from about HLB 8 to about HLB 16. In certain embodiments the combined calculated on the molar basis HLB of the emulsifier mixture is from about HLB 8 to about HLB 15. In certain embodiments the combined HLB calculated on the molar basis HLB of the mixture of the emulsifiers is from about HLB 10 to about HLB16. In certain embodiments the combined calculated on the molar basis HLB of the mixture of the emulsifiers is from about HLB 10 to about HLB 15. In certain embodiments the combined HLB calculated on the molar basis HLB of the mixture of the emulsifiers is from about HLB 10 to about HLB14. In certain embodiments the combined HLB calculated on the molar basis HLB of the mixture of the emulsifiers is from about HLB 10 to about HLB13. In certain embodiments the combined HLB calculated on the molar basis HLB of the mixture of the emulsifiers is from about HLB 8 to about HLB 12.

In certain embodiments, the pharmaceutical oil-in-water type emulsion composition comprises (i) from about 0.01% w/w to about 10% w/w, preferably from about 0.05% w/w to about 5% w/w, more preferably about 0.2% w/w to about 2% w/w of at least one cannabinoid; (ii) from about 0.005% w/w to about 10%, w/w, preferably from about 0.05% w/w to about 5% w/w, more preferably from about 0.01% w/w to about 2% w/w of at least one terpene or essential oil; (iii) from about 0.5% w/w to about 10% w/w or from about 2% w/w to about 5% w/w of at least two emulsifiers; (iv) from about 1% w/w to about 20% w/w, preferably from about 1% w/w to about 10% w/w, preferably from 2% w/w to 8% w/w of a triglyceride oil. Each possibility represents a separate embodiment of the invention.

In certain embodiments, the pharmaceutical emulsified cannabinoid composition having a plurality of particles having a mean particle size by number in the range of 0.01 to 10 microns. In certain embodiments, the pharmaceutical emulsified cannabinoid composition having of particles having a mean particle size in the range of 0.01 to 2 microns. In certain embodiments, the pharmaceutical emulsified cannabinoid composition having produce a plurality of particles having a mean particle size in the range of 0.01 to 1 micron. In certain embodiments, the cannabinoid emulsion composition consists of a plurality of particles having a mean particle size in the range of 0.01 to 0.8 microns or 0.6 microns or 0.4 microns.

In certain embodiments, the cannabinoid emulsion composition of this invention has a plurality of particles, wherein at least 90% of the particles have a size of 2 microns or less. In certain embodiments, the cannabinoid emulsion composition consists of a plurality of particles, wherein at least 90% of the particles have a size of 1 micron or less. In certain embodiments, the cannabinoid emulsion composition consists of a plurality of particles, wherein at least 95% of the particles have a size of 1 micron or less. In certain embodiments, the cannabinoid emulsion composition consists of a plurality of particles, wherein at least 98% of the particles have a size of 1 micron or less. In certain embodiments, the cannabinoid emulsion composition consists of a plurality of particles, wherein at least 99% of the particles have a size of 1 micron or less. In certain embodiments, the cannabinoid emulsion composition consists of a plurality of particles, wherein at least 99% of the particles have a size of 0.8 microns or less or 0.6 microns or less, or 0.4 microns or less.

In certain embodiments, the compositions of this invention further optionally comprise additional ingredients, selected from PGP inhibitors, intestinal PGP efflux inhibitors, absorption enhancers, viscosity modulating agents, antioxidants, stabilizing agents, fillers, glidants disintegration agents, coating and enteric-coating agents, microbial preserving agents, buffers and combinations thereof.

P-glycoprotein (PGP) inhibitors like Cytochrome P450/P-gp include any agent incorporated into the formulation matrix that inhibits pre-systemic hepatic first pass metabolism (i.e. first pass metabolism), such as d-alpha.-tocopheryl polyethylene glycol 1000 succinate, anise oil, cinnamon oil, coriander oil, grapefruit oil, lemon oil, orange oil, peppermint oil, ascorbyl palmitate, propyl gallate, piperin, curcumin, resveratrol, terpenes (essential oils) and various combinations thereof.

Intestinal PGP efflux inhibitors include any agent incorporated into the formulation matrix that inhibits PGP induced cellular efflux mechanisms (i.e. MDR), such as polyethoxylated castor oil derivatives, polyoxyethylene sorbitan monooleate, polyoxyethylene glycerides, herbal extracts such as, for example; piperin, ginger licorice, berberin, terpenes (essential oils) and various combinations thereof.

Absorption enhancers are selected from herbal extracts such as piperin, ginger extract, berberin, liquorice, quercetin, resveratrol, terpenes and vitamin E PEG 1000 succinate (d-.alpha.-tocopheryl polyethylene glycol 1000 succinate or TPGS) and mixtures thereof.

These optional ingredients can be used either alone or in combination with other ingredients to improve the chemical and physical properties of the oil-in-water type emulsion drug delivery systems and the cannabinoids or the terpenes chemical stability and shelf life.

Antioxidants include ascorbyl palmitate, butylated hydroxy anisole, butylated hydroxy toluene, propyl gallate, α-tocopherol, and γ-tocopherol, etc. The antioxidants that can be chosen include combinations of two or more agents described above, whereby ascorbyl palmitate and tocopherol provide optimal synergistic effects.

Furthermore, the dosage form may include viscosity modulating agents, stabilizing agents, fillers, glidants disintegration agents, coating and enteric-coating, microbial preserving agents, buffers, as known in the art, to produce the desired dosage form and manufacturability.

The present invention further provides, in some embodiments, a dosage form, comprising or consisting of any one of the compositions described in this invention.

The term “dosage form” in this context denotes any form of the composition of this invention that contains an amount of at least one cannabinoid sufficient to achieve at least a partial therapeutic effect with a single administration.

In certain embodiments, the dosage form is an oral dosage form. In certain embodiments, the dosage form is a rectal dosage form. In certain embodiments, the dosage form is a nasal dosage form. In certain embodiments, the dosage form is mucosal dosage form. In certain embodiments, the dosage form is a vaginal dosage form. In certain embodiments, the dosage form is a topical dosage form. In certain embodiments, the dosage form is an otic dosage form.

In certain embodiments, the dosage form is formulated as a liquid, a syrup, an enema, an emulsion or a lotion. Each possibility represents a separate embodiment of the invention. In certain embodiments, the dosage form is formulated for mucosal delivery. The term “mucosal delivery” refers to the delivery to a mucosal surface, including nasal, pulmonary, vaginal, rectal, urethral, sublingual and buccal delivery. In certain embodiments, the dosage form is formulated in a candy, toffee, dragee, chocolate, cookie or lozenge.

According to the principles of the present invention, the emulsified cannabinoid compositions dosage form comprises a “cannabis active ingredient” (i.e. Cannabis-extracted and purified cannabinoid or synthetic cannabinoid or cannabis extract), optionally a terpene, an oil, an emulsifier or emulsifiers and optionally inactive ingredients. The advantages of the pharmaceutical composition over known cannabis compositions are manifold and include: (a) high oral bioavailability (b) stable emulsion product of oil-in-water type, enabling stable formulations and combinations of desired cannabinoids and optionally terpenes, in simple and common production methods and machinery (c) ready to use and easy to swallow and administer to all segments of population including infants, toddlers, adults and elderly, for successful patient compliance and effective medication (d) good taste and no bitter taste (e) a high strength cannabinoid, pleasant and ready to use syrup.

In an embodiment, the cannabis emulsion composition in a concentrated form, without water, is a self-emulsifying system, which when coming in contact with an aqueous medium such as saline or simulated intestinal fluids or gastro intestinal fluids, at body temperature and mild agitation, forms a fine dispersion with mean particle size below 20 microns, below 10 microns, below 5 microns, below 2 microns or below 1 micron.

The concentrated composition is essentially waterless or anhydrous, which means essentially devoid of water. The term “waterless” or “anhydrous” has the conventional use and meaning for a composition which does not comprise water. In some embodiment, the composition is devoid of water or has less than 5 percent of water on a weight basis. In some embodiment, the composition is devoid of water or has less than 5 percent of water on a weight basis. In some embodiments, the composition is devoid of water or has less than 4 percent of water on a weight basis. In some embodiments, the composition is devoid of water or has less than 3 percent of water on a weight basis. In some embodiments, the composition is devoid of water or has less than 2 percent of water on a weight basis. In some embodiments, the composition is devoid of water or has less than 1 percent of water on a weight basis. In some embodiments, the composition is devoid of water or has less than 0.5 percent of water on a weight basis. In some embodiments, the composition is devoid of water or has less than 0.1 percent of water on a weight basis.

In some embodiments, optional ingredients of the composition include absorption enhancers, such as cytochrome P450 metabolic inhibitors, P-glycoprotein efflux inhibitors and intestinal epithelial cells tight junction temporal openers.

In some embodiments, a functional inactive ingredient maybe optionally be added, such as a colorant or an antioxidant or a chelating agent or a microbial preservative or a viscosity modifier, a pH modifying agent, a buffer, a melting point modifier, an anti-microbial agent, a suspending agent or combinations thereof.

The present invention further provides, in another aspect, a composition as described above, or a dosage form as described above, for use in a method of treating a cannabinoid-responsive symptom, disease or disorder.

The phrase “cannabinoid-responsive symptom, disease or disorder” or “cannabinoid treatable disorders used herein refers to any symptom, disease or disorder which is amenable to therapeutic benefit by a cannabinoid, by a mixture of cannabinoids, or by extracts of Cannabis.

In certain embodiments, the cannabinoid-responsive symptom, disease or disorder is selected from the group consisting of: pain associated with cancer, neuropathic pain and HIV-associated sensory neuropathy; side effects of chemotherapy including nausea; symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anxiety, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, insomnia, fatigue, sleep apnea, digestive diseases, inflammatory bowel diseases, collagen-induced arthritis, atherosclerosis, auto immune diseases and dystonia and geriatric syndromes. Each possibility represents a separate embodiment of the invention.

Terpenes are fragrant oils that give cannabis its aromatic diversity. These oils are secreted in the flower's sticky resin glands, the same ones that produce THC, CBD, and other cannabinoids. Terpenes are by no means unique to cannabis; they can be found in many other herbs, fruits, and plants as well.

Secreted in the same glands that produce cannabinoids like THC and CBD, terpenes are the pungent oils that color cannabis varieties with distinctive flavors like citrus, berry, mint, and pine. Over 100 different terpenes have been identified in the cannabis plant, and every strain tends toward a unique terpene type and composition. THC binds to cannabinoid receptors concentrated heavily in the brain where psychoactive effects are produced. Some terpenes also bind to these receptor sites and affect their chemical output. Others can modify how much THC passes through the blood-brain barrier. Their hand of influence even reaches to neurotransmitters like dopamine and serotonin by altering their rate of production and destruction, their movement, and availability of receptors.

The effects these mechanisms produce vary from terpene to terpene; some are especially successful in relieving stress, while others promote focus and acuity. Myrcene, for example, induces sleep whereas limonene elevates mood. There are also effects that are imperceptible, like the gastroprotective properties of caryophyllene.

TABLE 1
Most common Cannabis terpenes, their effects and medical value.

| Terpene | Effects | Medical Value | Also Found In |
|---|---|---|---|
| Alpha-Pinene, | Alertness, memory | Asthma, antiseptic | Pine needles, |
| Beta-Pinene | retention, counteracts | | rosemary, basil, |
| | some THC effects | | parsley, dill |
| Myrcene | Sedating “couchlock” | Antioxidant, anti- | Mango, lemongrass, |
| | effect, relaxing | carcinogenic; good for | thyme, hops |
| | | muscle tension, sleeplessness, | |
| | | pain, inflammation, | |
| | | depression | |
| Limonene | Elevated mood, | Antifungal, anti-bacterial, | Fruit rinds, |
| | stress relief | anti-carcinogenic, dissolves | rosemary, juniper, |
| | | gallstones, mood-enhancer; | peppermint |
| | | may treat gastrointestinal | |
| | | complications, heartburn, | |
| | | depression | |
| Caryophyllene | No detectable | Gastroprotective, anti- | Black pepper, |
| | physical effects | inflammatory; good for | cloves, cotton |
| | | arthritis, ulcers, autoimmune | |
| | | disorders, and other | |
| | | gastrointestinal complications | |
| Linalool | Anxiety relief and | Anti-anxiety, anti-convulsant, | Lavender |
| | sedation | anti-depressant, anti-acne | |

Some cannabinoids and terpenes have similar pharmacological activities, and are working in synergy or entourage. Insomnia, which is sleep disorder, anxiety and depression as well as pain of various types are some of the common diseases or disorders where the combination of cannabinoids and terpenes are very useful. Cannabis plants are producing cannabinoids as well as terpenes which are volatile and give the cannabis plants and its various extracts their unique smell and human recognition.

Cannabis is an effective sleep-inducing agent. It is estimated that one in three people suffer from some form of insomnia. Insomnia can cause an increased number of accidents, absenteism from work, and an overall decreased quality of life. 95% of people who have insomnia take medication to help them with sleep, and sleep medications can become very addictive.

Cannabis is a natural alternative to pharmaceuticals for insomnia relief. A study published in 1973 in Human Pharmacology found that subjects had a significant decrease in the amount of time it took them to fall asleep when given THC. A study published in 1981 also found that when patients with insomnia took a 160 mg dose of CBD, their length of sleep increased compared to those who took a placebo. Psychopharmacologia. 1973 Dec. 20; 33(4):355-64. (-) Delta 9 THC as an hypnotic. An experimental study of three dose levels. Cousens K, DiMascio A. J Clin Pharmacol. 1981 August-September; 21(8-9 Suppl):417S-427S. Hypnotic and antiepileptic effects of cannabidiol. Carlini E A, Cunha J M.

THC, CBN or marijuana (cannabis) before bed also appears to reduce the density of rapid eye movements during rapid eye movement (REM) sleep. Interestingly, less REM density has been linked to more restful sleep.

Terpenes are used for centuries to treat insomnia. Myrcene and Limonene having sedative effects and combinations of cannabinoids and terpenes are apparently having “entourage” effect or synergistic effect. THC, CBD and CBN are major documented sedative or sleep-inducing cannabinoids. Cannabinoids were documented to reduce sleep REM, inducing sleep and promoting better and longer sleep with reduced dreams which is beneficial also to anxiety and post-traumatic stress disorder (PTSD) patients. frequent cannabis users dream much less often than others. This is linked to marijuana's impact on REM sleep, since most dreaming occurs during this portion of one's sleep cycle.

People with PTSD may also benefit from marijuana use when it comes to sleep. When a person has experienced a traumatic event, they may suffer from nightmares that interfere with their sleep. Marijuana use is common among those with post-traumatic stress disorder (PTSD), and patients report using it to help them sleep. Using a synthetic THC as a treatment for PTSD-related nightmares, it was found that patients slept longer, had higher sleep quality, and experienced fewer daytime flashbacks when using the drug.

Another common reason people seek relief with cannabis is to reduce pain at night and improve sleep. The combination of pain and poor sleep can have serious effects on a person's wellbeing. Marijuana is known for its ability to relieve pain and to help with insomnia. Likewise, research shows that many people who use marijuana for chronic pain find that it also improves their quality of sleep.

Parkinson's disease is associated with a very unusual form of sleep disturbance. The disturbances are collectively referred to as REM sleep behavior disorder. During REM sleep, our bodies lose muscle tone which prevents us from physically acting out our dreams in bed. However, in REM sleep behavior disorder, the body no longer loses muscle tone, causing patients to act out their dreams. This may involve punching, pushing, yelling and swearing while sleeping. In a series of case studies, doctors observed improvements in sleep quality in patients who used CBD. They found that doses of 75-300 mg of CBD taken daily could reduce or eliminate entirely any symptoms of REM sleep behavior disorder.

Cannabis strains high in Myrcene and Linalool, usually of Indica type, are associated with sedation and better sleep.

Chronic pain is one of the most common conditions treated with medical marijuana. Similar to over-the-counter painkillers such as aspirin and ibuprofen, marijuana can reduce inflammation and pain associated with inflammation.

THC is a compound believed to reduce pain. It has been found to be effective in a variety of conditions that cause pain, including arthritis, migraine, multiple sclerosis and cancer. A 2015 clinical review examined 6 different trials with a total of 325 patients, and concluded that marijuana can be an effective treatment for patients with chronic pain. Hill at al, 2015, JAMA. 2015; 313(24):2474-2483. Medical Marijuana for Treatment of Chronic Pain and Other Medical and Psychiatric Problems. Hill at al, Cannabis and Pain: A Clinical Review, Cannabis and Cannabinoid Research Volume 2.1, 2017

Marijuana may be beneficial to those with certain liver disorders. In the case of liver fibrosis (scarring), a 2011 study highlighted the benefits of CBD, a cannabinoid found in marijuana. Specifically, CBD may contribute to the cell death of hepatic stellate cells that contribute to liver scarring. This suggests that CBD may reduce the extent of scarring in the liver when it is damaged.

Marijuana use in patients with cancer is becoming increasingly common. One of the most commonly reported benefits is the reduction of nausea and vomiting for patients in chemotherapy. Besides reducing the unpleasant symptoms of chemotherapy, marijuana shows potential as a cancer therapy itself. In mice and rat models, researchers have found that THC and other cannabinoids can trigger cell death in many types of cancer cells. In 2007, researchers at Harvard University found that THC may reduce the size of human lung tumors implanted into rats and mice. The reduction in tumor mass and volume were found to be as high as 50%, and the reduction of cancerous lesions in the lungs was around 60%.

Using marijuana may be beneficial for patients living with different forms of inflammatory bowel disease, including Crohn's disease and ulcerative colitis. Observational studies indicate that using marijuana can result in an improvement in symptoms, as well as a reduction in the use of standard medications. In a 2014 study, researchers found that IBD patients who used marijuana showed greater improvements in symptoms than those who received a placebo. The study found that of the 11 participants given marijuana, 5 were able to achieve complete remission of the disease. Inflammation: Tambe Y, Tsujiuchi H, Honda G, et al. 1996. Gastric cyto-protection of the non-steroidal anti-inflammatory sesquiterpene, beta-caryophyllene. Planta Med, 62:469-70.

Geriatric syndrome (GS) are multifactorial, and have shared risk factors-including older age, cognitive impairment, functional impairment, and impaired mobility-were demonstrated across the common geriatric syndromes of pressure ulcers, incontinence, falls, functional decline, and delirium. The term “geriatric syndrome” is used to capture those clinical conditions in older persons that do not fit into discrete disease categories. Many of the most common conditions cared for by geriatricians, including delirium, falls, frailty, dizziness, syncope and urinary incontinence, are classified as geriatric syndromes.

Cannabis and cannabinoids effects are useful for geriatric syndrome patients while the side effects of cannabis are trivial in comparison to drug products medication. Cannabinoids are alleviating pain, specifically skeletal and neuropathic pains, are bone builders, increasing appetite and are sedatives. However not all the cannabinoids and cannabis extracts are the same and practically many cannabis extracts are having opposite effects. Some are sedatives, and some are alerting. No single cannabinoid is as effective as the mixture of various cannabinoids with terpenes, termed “entourage effect”.

Direct association between frailty and elevated circulating levels of interleukin (IL)-6, a proinflammatory cytokine, was first observed in community-dwelling older adults. A large number of studies in many cohorts of older adults and under various care settings have since provided evidence supporting the role of chronic inflammation and immune activation in the pathogenesis of the frailty syndrome (https://www.ncbi.nlm.nih.gov/pmc/articles/PMC3964027/)

Chronic pain is the most common reason that physicians recommend marijuana in the states where it is allowed for medical use. Marijuana relieves pain without over sedation. Marijuana allows pain patients to use fewer narcotics and fewer nonprescription, or over-the-counter, pain medications.

Marijuana is far safer than anti-depressant medications, tranquilizers and alcohol—alternatives the elderly otherwise seek out to deal with these issues. Marijuana is also an appetite stimulant—it causes “the munchies.” This can help an aging population to maintain an appropriate weight and fight off cachexia, the wasting syndrome. The frail elderly can be turned into a more robust elderly with marijuana or cannabinoids therapy.

In some embodiments, the present invention provides a composition comprising cannabis or at least one cannabinoid for treatment of the elderly population and geriatric syndrome patients. Specific and highly beneficial cannabinoid compositions are provided for treatment and alleviation of the complex conditions and plethora of the geriatric syndromes manifestations as well as improvement of quality of life.

Cannabis is used to treat various diseases that are very common in elderly population, however, currently there is no accepted cannabis standardization and cannabis mode of administration that is specific and suited for the elderly GS patients. Elderly are using cannabis by smoking, vaping, edible cookies and the like, a mode of administration which cannot be easily monitored.

The current invention is useful in improving elderly and GS patients's quality of life, reducing use of drugs, the poly-pharmacy, improving wellbeing, insomnia, alertness and personal activity, reducing intensity of inflammation and improving the cognition.

It is highly needed to provide a cannabis or cannabinoid anti-inflammatory and pain control composition that will provide the cannabis effects without reducing the elderly patient alertness during the day time and a different and matching cannabis or cannabinoid composition for the night time that will be relaxing, anti-depressant and provide good sleep and alleviate insomnia. Cannabis is effective in alleviating the geriatric syndrome by improving sleep, increasing appetite, reducing spasticity and pain, anti-inflammation, reduce depression, improve osteoporosis, reduce polypharmacy the use of too many drugs with tremendous side effects reducing quality of life and numerous identified and unidentified contra indications, and alleviating delirium and frailty.

The day time composition of this invention comprises at least about 20 mg cannabidiol (CBD) and at least about 1 mg of at least one alerting terpene. The CBD comprising at least 90% of total cannabinoids, and whereas the ration of CBD to psychoactive cannabinoids tetra hydro cannabinol (THC) is at least about 10, and preferable about 20 or about 50 or about 100.

The night time composition comprises about 2 mg to about 10 mg of sedating cannabinoids, selected from cannabis plant extract or purified cannabinoids or derivatives with at least 2 mg of THC and/or CBN, and the at least one terpene about 0.05 mg of at least one sedative terpene.

In some embodiments, there is provided an alerting composition of this invention, comprising an alerting essential oil or combinations thereof, selected from Limonene, Caryophyllene, Pinene, Rosmarinus oil, citrus oil and combinations thereof.

According to some embodiments, the terpenes selected for treating depression, anxiety, PTSD, psychiatric disorders such as schizophrenia or geriatric syndrome are of the alerting type such as Limonene, alfa and beta pinene, orange terpenes, thymol, isoborneol, and isoeugenol, and avoiding the sedating type terpenes such as myrcene, linalool, linalyl acetate, alfa terpineol, terpinolene and citronellal, sandalwood, lavender, valerian and neroli oils as examples. Example of alerting cannabis strains are for example: Harlequin, Charlotte's Web, ACDC, Avidekel, and Remedy, all are trade names and trademarks.

In certain embodiments, the pharmaceutical composition of this invention comprises or consists of one of the formulations presented in Tables 2-12. Each table represents a separate embodiment of the invention.

### EXAMPLES

### Example 1

TABLE 2
Compositions for sleep aid, insomnia and sedation

| | Formula # | | | | | |
|---|---|---|---|---|---|---|
| | 1A | 1B | 1C | 1D | 1E | 1F |
| Ingredient | % W/W | % W/W | % W/W | % W/W | % W/W | % W/W |
| THC | 0.06 | 0.06 | 0.06 | — | 0.06 | 0.06 |
| CBN | — | 0.02 | — | 0.02 | — | 0.02 |
| Afghan indica pure extract | — | — | — | 0.05 | — | — |
| Myrcene | 0.05 | — | — | — | 0.05 | 0.05 |
| Linalool | 0.05 | — | — | — | 0.05 | — |
| Lavender oil | — | 0.10 | — | 0.05 | — | — |
| Valerian oil | — | — | 0.10 | — | — | 0.1 |
| Capric/caprylic triglyceride | 4.0 | 4.0 | 4.0 | 4.0 | 4.0 | 4.0 |
| Sucrose stearate | 1.5 | 1.5 | 1.5 | 1.2 | 0.8 | 0.8 |
| Tocopheryl PEG 1000 | 1.5 | 1.5 | 1.5 | 1.2 | 0.8 | 0.8 |
| succinate | | | | | | |
| PEG-35 Castor Oil | 1.5 | 1.5 | 1.5 | 1.2 | 0.8 | 0.8 |
| Anti-oxidants | 0.05 | 0.05 | 0.05 | 0.05 | 0.05 | 0.05 |
| Sweetener | 0.5 | 0.5 | 0.5 | 0.5 | 0.5 | 0.5 |
| Microbial preservative | 0.05 | 0.05 | 0.05 | 0.05 | 0.05 | 0.05 |
| Purified water | To 100 | To 100 | To 100 | To 100 | To 100 | To 100 |
| Max particle size nm | About 600 | About 600 | About 600 | About 600 | About 800 | About 800 |
| Mean particle size nm | 85 | Below 200 | Below 200 | Below 200 | Below 200 | Below 200 |
| Appearance | Translucent | Translucent | Translucent | Translucent | Translucent | Translucent |
| Type | Nano- | Nano- | Nano- | Nano- | Nano- | Nano- |
| | emulsion | emulsion | emulsion | emulsion | emulsion | emulsion |
| Centrifugation test | Passed | Passed | Passed | Passed | Passed | Passed |

Each formulation was subjected to a centrifugation stability test at 3,500 RPM for five minutes in an Eppendorf™ transparent plastic tube. The emulsion was examined visually and if no sign of separation, segregation or creaming was observed, the formulation passed the test.

The cannabis extract composition in Table 2 was produced by an ethanol extraction of decarboxylated plant material, and comprises about 60% by weight THC, about 20% by weight CBD, and about 4% by weight terpenes.

The “Largest detected size” of the particles was determined by placing a drop on bearing glass and covered with top glass, and the sample was immediately examined with light microscope (Nikon, Eclipse™ E200) magnification ×200 or ×400 with calibrated scale.

The “Mean particle size” of the particles was also determined by the DLS method using a Malvern Nanosizer™. The sample was diluted 1:200 with distilled water and slowly rotated. The test was performed according to Malvern Nanosizer™ instructions, and further dilutions were performed as needed and indicated by the instrument. The “mean particle size” was calculated based on the intensity and number distribution of all particles. The mean particle size measured by DLS or by light microscope and presented in the various tables, is for about 99% or the number of particles.

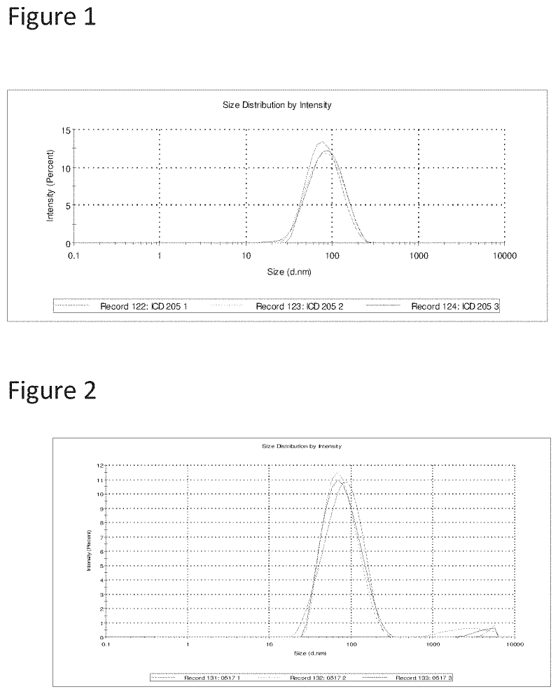

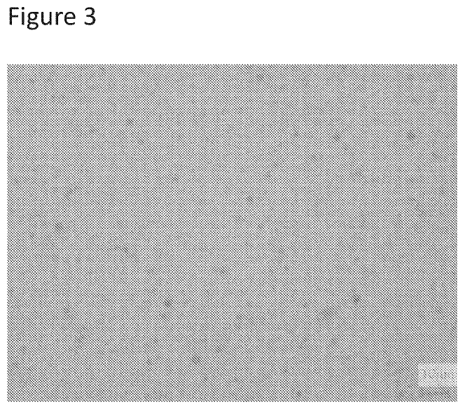

All cannabinoid emulsion compositions of tables 1-11 were tested with light microscope for particle size. However, light microscope is limited to high wavelength and nano-particles that are below the visible wavelength are not seen with light microscope. The Malvern™ nanosizer uses diffraction laser spectroscopy (DLS) in order to measure particle size below the visible light wavelength. In case of sub-micron and nano-size oily particles of the internal phase of the emulsion, the light microscope can detect only the fraction of particles that are within and above the visible light wavelength, if there are such particles. All of the compositions of tables 1-11 are nano-emulsions, and therefore the mean particle size could not be detected by light microscope. FIG. 1 shows DLS results of a composition 1A which is a nano-emulsion and no particles were detected by the light microscope. FIG. 2 shows the DLS results of composition 517 of table 11, wherein two populations were detected, nano-size and micron-size. FIG. 3 shows the population of larger particles only of composition 517 of table 11, as seen with light microscope and magnification ×400 and the results are in agreement between the two methods of detection of particle size, the DLS and the light microscope.

### Example 2

TABLE 3
Alerting cannabinoids and terpenes compositions for day time oral medication

| | Formula # | | | | | | | |
|---|---|---|---|---|---|---|---|---|
| | 2A | 2B | 2C | 2D | 2E | 2F | 2G | 2H |
| Ingredient | % W/W | % W/W | % W/W | % W/W | % W/W | % W/W | % W/W | % W/W |
| CBD | 0.5 | 1.0 | 2.0 | 1.0 | 0.5 | 1.0 | — | — |
| Harlequin pure extract | — | — | — | — | — | — | 2.0 | 4.0 |
| Limonene | 0.05 | — | — | 0.05 | — | — | 0.05 | 0.05 |
| Pinene | 0.05 | — | — | 0.05 | — | — | 0.05 | 0.05 |
| Thymol | — | 0.10 | — | — | 0.10 | — | — | — |
| Orange essential oils | — | | 0.10 | — | — | 0.10 | — | — |
| Capric/caprylic | 4.0 | 6.0 | 4.0 | 6.0 | 4.0 | 6.0 | 4.0 | 6.0 |
| triglyceride | | | | | | | | |
| Sucrose stearate | 1.2 | 1.2 | 1.2 | 1.5 | — | — | 1.0 | 1.0 |
| Tocopheryl PEG 1000 | 1.2 | 1.2 | 1.2 | — | 1.5 | 1.5 | 1.0 | 1.0 |
| succinate | | | | | | | | |
| PEG-35 Castor Oil | 1.2 | 1.2 | 1.2 | 1.5 | 1.5 | 1.5 | 1.0 | 1.0 |
| Anti-oxidants | 0.05 | 0.05 | 0.05 | 0.05 | 0.05 | 0.05 | 0.05 | 0.05 |
| Sweetener | 2.5 | 2.5 | 3.0 | 3.0 | 3.5 | 3.5 | 4.0 | 4.0 |
| Microbial preservative | 0.05 | 0.05 | 0.05 | 0.05 | 0.05 | 0.05 | 0.05 | 0.05 |
| Purified water | To 100 | To 100 | To 100 | To 100 | To 100 | To 100 | To 100 | To 100 |

All emulsion cannabinoid compositions of table 2 passed the centrifugation stability test, are translucent, with max particle size below 1 micron and mean particle size from 100 nm to 500 nm

### Example 3

TABLE 4
Anti-inflammatory compositions of cannabinoids and terpenes

| | Formula # | | | | | | | | |
|---|---|---|---|---|---|---|---|---|---|
| | 3A | 3B | 3C | 3D | 3E | 3F | 3G | 3H | 3I |
| Ingredient | % W/W | % W/W | % W/W | % W/W | % W/W | % W/W | % W/W | % W/W | % W/W |
| CBD | 2.0 | — | — | 2.0 | — | — | 4.0 | — | |
| CBDA | — | 2.0 | — | — | 2.0 | — | — | 4.0 | |
| THCA | | | 2.0 | | | 2.0 | | | 4.0 |
| Caryophyllene | 0.5 | 0.2 | — | 0.5 | 0.2 | — | 0.2 | 0.4 | 0.2 |
| Humulene | — | 0.2 | 0.2 | — | 0.2 | 0.2 | 0.2 | — | — |
| Myrcene | — | — | 0.1 | — | — | 0.1 | 0.2 | — | 0.2 |
| Pinene | — | — | 0.1 | — | — | 0.1 | 0.2 | 0.4 | 0.2 |
| Capric/caprylic | 6.0 | 6.0 | 6.0 | 6.0 | 6.0 | 8.0 | 8.0 | 8.0 | 8.0 |
| triglyceride | | | | | | | | | |
| Sucrose stearate | 1.5 | 1.5 | 1.5 | 1.5 | 1.5 | 2.0 | 2.0 | 2.0 | 2.0 |
| Tocopheryl PEG 1000 | 1.5 | 1.5 | 1.5 | 1.5 | 1.5 | 2.0 | 2.0 | 2.0 | 2.0 |
| succinate | | | | | | | | | |
| PEG-35 Castor Oil | 1.5 | 1.5 | 1.5 | 1.5 | 1.5 | 2.0 | 2.0 | 2.0 | 2.0 |
| Alkyl Acrylate Cross | 0.5 | — | — | 0.5 | — | — | 0.5 | — | — |
| polymer (Pemulene ™ | | | | | | | | | |
| TR2) | | | | | | | | | |
| Anti-oxidants | 0.05 | 0.05 | 0.05 | 0.05 | 0.05 | 0.05 | 0.05 | 0.05 | 0.05 |
| Sweetener | 4.0 | 4.0 | 4.0 | 4.0 | 4.0 | 4.0 | 4.0 | 4.0 | 4.0 |
| Microbial preservative | 0.05 | 0.05 | 0.05 | 0.05 | 0.05 | 0.05 | 0.05 | 0.05 | 0.05 |
| Purified water | To 100 | To 100 | To 100 | To 100 | To 100 | To 100 | To 100 | To 100 | To 100 |

All emulsion cannabinoid composition of table 3 passed the centrifugation test, are translucent, with max particle size below 1 micron and mean particle size from 100 nm to 500 nm.

### Example 4

TABLE 5
Anti-depression, anti-anxiety and post-traumatic stress disorder (PTSD)
compositions comprising cannabinoids and terpenes

| | Formula # | | | | | | | | |
|---|---|---|---|---|---|---|---|---|---|
| | 4A | 4B | 4C | 4D | 4E | 4F | 4G | 4H | 4I |
| Ingredient | % W/W | % W/W | % W/W | % W/W | % W/W | % W/W | % W/W | % W/W | % W/W |
| CBD | 2.0 | — | — | 2.0 | — | — | 4.0 | — | |
| Cannabis extract | — | 2.0 | — | — | 2.0 | — | — | 4.0 | |
| CBD:THC 20:1 | | | | | | | | | |
| Cannabis extract | — | — | 0.5 | — | — | 0.5 | — | — | 0.5 |
| CBDA:THC 1:1 | | | | | | | | | |
| Caryophyllene | 0.5 | 0.2 | — | 0.5 | 0.2 | — | 0.2 | 0.4 | 0.2 |
| Humulene | — | 0.2 | 0.2 | — | 0.2 | 0.2 | 0.2 | — | — |
| Myrcene | — | — | 0.1 | — | — | 0.1 | 0.2 | — | 0.2 |
| Pinene | — | — | 0.1 | — | — | 0.1 | 0.2 | 0.4 | 0.2 |
| Capric/caprylic | 6.0 | 6.0 | 6.0 | 6.0 | 6.0 | 8.0 | 8.0 | 8.0 | 2.0 |
| triglyceride | | | | | | | | | |
| Sucrose stearate | 1.2 | 1.0 | 0.8 | 1.5 | 1.5 | 1.5 | 1.5 | 1.5 | 0.8 |
| Tocopheryl PEG | 1.2 | 1.0 | 0.8 | 1.5 | 1.5 | 1.5 | 1.5 | 1.5 | 0.8 |
| 1000 succinate | | | | | | | | | |
| PEG-35 Castor Oil | 1.2 | 1.0 | 0.8 | 1.5 | 1.5 | 1.5 | 1.5 | 1.5 | 0.8 |
| Anti-oxidants | 0.05 | 0.05 | 0.05 | 0.05 | 0.05 | 0.05 | 0.05 | 0.05 | 0.05 |
| Sweetener | 4.0 | 4.0 | 4.0 | 4.0 | 4.0 | 4.0 | 4.0 | 4.0 | 4.0 |
| Microbial | 0.05 | 0.05 | 0.05 | 0.05 | 0.05 | 0.05 | 0.05 | 0.05 | 0.05 |
| preservative | | | | | | | | | |
| Purified water | To 100 | To 100 | To 100 | To 100 | To 100 | To 100 | To 100 | To 100 | To 100 |

All emulsion cannabinoid composition of table 3 passed the centrifugation test, are translucent, with max particle size below 1 micron and mean particle size from 500 nm to 200 nm.

### Example 5

TABLE 6
Stable cannabinoid and terpene emulsions compositions comprising increasing
concentrations of Medium Cain Triglycerides (MCT oil),

| Formula # | 5A | 5B | 5C | 5D | 5E |
|---|---|---|---|---|---|
| Ingredient | % W/W | % W/W | % W/W | % W/W | % W/W |
| CBD | 0.5 | 0.5 | 0.5 | 0.5 | 0.5 |
| Rosmarinus oil | 0.1 | 0.1 | 0.1 | 0.1 | 0.1 |
| Capric/caprylic triglyceride | 2.0 | 4.0 | 6.0 | 8.0 | 10.0 |
| Sucrose stearate | 0.8 | 0.8 | 1.5 | 2.0 | 2.0 |
| Tocopheryl PEG 1000 succinate | 0.8 | 0.8 | 1.5 | 2.0 | 2.0 |
| PEG-35 Castor Oil | 0.8 | 0.8 | 1.5 | 2.0 | 2.0 |
| Anti-oxidants | 0.05 | 0.05 | 0.05 | 0.05 | 0.05 |
| Sweetener | 4.0 | 4.0 | 4.0 | 4.0 | 4.0 |
| Microbial preservative | 0.05 | 0.05 | 0.05 | 0.05 | 0.05 |
| Purified water | To 100 | To 100 | To 100 | To 100 | To 100 |
| Max particle size nm | 500 | 600 | 800 | 1,000 | 2,000 |
| Mean particle size nm | 54 | 85 | 92 | 117 | 114 and |
| | | | | | 1520 |
| Appearance | Translucent | Translucent | Translucent | Translucent | Light milky |
| | | | | | emulsion |
| Type | Nano | Nano | Nano | Nano | Sub-micron |
| | emulsion | emulsion | emulsion | emulsion | emulsion |
| Centrifugation test | Passes | Passes | Passes | Passes | Passes |

### Example 6

TABLE 7
Stable emulsion compositions comprising various triglyceride oils

| | Formula # | | | | | | | |
|---|---|---|---|---|---|---|---|---|
| | 6A | 6B | 6C | 6D | 6E | 6F | 7G | 7H |
| Ingredient | % W/W | % W/W | % W/W | % W/W | % W/W | % W/W | % W/W | % W/W |
| Cannabis extract 80% | 0.5 | 0.5 | 0.5 | 0.5 | 0.5 | 0.5 | 0.5 | 0.5 |
| cannabinoids | | | | | | | | |
| THC:CBD 1:1 | | | | | | | | |
| Rosmarinus oil | 0.1 | 0.1 | 0.1 | 0.1 | 0.1 | 0.1 | 0.1 | 0.1 |
| Capric/caprylic | 12.0 | — | — | — | 4.5 | 6.0 | 6.0 | 2.0 |
| triglyceride | | | | | | | | |
| Olive oil | — | 6.0 | 6.8 | 5.5 | — | — | — | — |
| Hydrogenated castor | — | — | — | — | 3.5 | — | — | 2.0 |
| oil | | | | | | | | |
| Sucrose stearate | 2.0 | 0 | | — | — | 1.8 | 1.8 | 1.2 |
| Tocopheryl PEG 1000 | 2.0 | 0 | | — | 1.5 | 1.8 | 1.8 | 1.2 |
| succinate | | | | | | | | |
| PEG-35 Castor Oil | 2.0 | 4.0 | 5.6 | 2.13 | 4.7 | 1.8 | 1.8 | 1.2 |
| Polyglyceryl-3 dioleate | — | 4.0 | 5.6 | 2.13 | 2.9 | | | |
| Polysorbate 80 | == | 4.0 | — | 1.22 | — | | | |
| Alkyl acrylate | 0.8 | — | — | — | — | — | 0.4 | 0.4 |
| crosspolymer | | | | | | | | |
| (Pemulene ™ TR2) | | | | | | | | |
| Anti-oxidants | 0.05 | 0.05 | 0.05 | 0.05 | 0.05 | 0.05 | 0.05 | 0.05 |
| Sweetener | 4.0 | 4.0 | 4.0 | 4.0 | 4.0 | 4.0 | 4.0 | 4.0 |
| Microbial preservative | 0.05 | 0.05 | 0.05 | 0.05 | 0.05 | — | — | — |
| Purified water | To 100 | To 100 | To 100 | To 100 | To 100 | 20 | — | 20 |
| Sucrose syrup USP | — | — | — | — | — | To 100 | To 100 | |
| Silan (Dates honey) | — | — | — | — | — | — | | To 100 |

All emulsified cannabinoid composition of table 6 passed the centrifugation test and were translucent, with max particle size below 1 micron and mean particle size from 500 nm to 200 nm.

### Example 7

TABLE 8
Stable emulsion compositions comprising at least two emulsifiers

| Formula # | 7A | 7B | 7C | 7D | 7E |
|---|---|---|---|---|---|
| Ingredient | % W/W | % W/W | % W/W | % W/W | % W/W |
| CBD | 0.5 | 0.5 | 0.5 | 0.5 | 0.5 |
| Rosmarinus oil | 0.1 | 0.1 | 0.1 | 0.1 | 0.1 |
| Capric/caprylic | 4.0 | 4.0 | 4.0 | 4.0 | 4.0 |
| triglyceride | | | | | |
| Sucrose stearate | 1.2 | 0 | 2.0 | 0 | 0 |
| Tocopheryl PEG 1000 | 0 | 2.0 | 2.0 | 1.2 | 0 |
| succinate | | | | | |
| PEG-35 Castor Oil | 1.2 | 2.0 | 0 | 0 | 1.2 |
| Polysorbate 80 | 0 | 0 | 0 | 0 | 0 |
| Span 80 | 0 | 0 | 0 | 1.2 | 1.2 |
| Anti-oxidants | 0.05 | 0.05 | 0.05 | 0.05 | 0.05 |
| Sweetener | 4.0 | 4.0 | 4.0 | 4.0 | 4.0 |
| Microbial preservative | 0.05 | 0.05 | 0.05 | 0.05 | 0.05 |
| Purified water | To 100 | To 100 | To 100 | To 100 | To 100 |
| Max particle size nm | 5,000 | 2,000 | 2,000 | 2,000 | 1,000 |
| Mean particle size nm | 85 and | 84 and | 104 and | Below | Below |
| | 4032 | 1360 | 1484 | 600 | 600 |
| Appearance | Translucent | Translucent | Translucent | Translucent | Translucent |
| Type | Submicron | Submicron | Submicron | Submicron | Submicron |
| Centrifugation | Pass | Pass | Pass | Pass | Pass |

### Example 8

TABLE 9
Stable sub-micron emulsion compositions comprising cannabinoids and
various oils and oils which are solid at room temperature.

| | Formula # | | | | | |
|---|---|---|---|---|---|---|
| | 8A | 8B | 8C | 8D | 8E | 8F |
| Ingredient | %W/W | % W/W | % W/W | % W/W | % W/W | % W/W |
| CBD | 0.5 | 0.5 | 0.5 | 0.5 | 0.5 | 0.5 |
| Rosmarinus oil | 0.1 | 0.1 | 0.2 | 0.1 | 0.1 | 0.1 |
| Capric/caprylic triglyceride | 4.0 | — | — | 2.0 | 5.0 | 2.0 |
| Olive oil | — | 4.0 | — | — | — | — |
| Sesame oil | — | — | 6.0 | — | — | — |
| Hydrogenated castor oil | — | — | — | 2.0 | 3.0 | 2.0 |
| Behenyl alcohol | — | — | — | — | — | 2.0 |
| Sucrose stearate | 1.2 | — | — | 1.2 | — | — |
| Tocopheryl PEG 1000 succinate | 1.2 | 1.5 | 4.0 | 1.2 | 1.5 | 3.0 |
| PEG-35 Castor Oil | 1.2 | 3.0 | 4.0 | 1.2 | 4.0 | 3.0 |
| Polyclycerol-3 dioleate | — | 3.0 | 4.0 | — | 3.0 | 3.0 |
| Polysorbate 80 | — | — | — | | | |
| Xanthan gum (Xantural ™ | 1.2 | — | — | — | — | — |
| 3000) | | | | | | |
| Microcrystalline cellulose | — | 2.0 | — | 1.0 | — | — |
| (Avicel ™ 611 | | | | | | |
| Alginate | — | — | — | 0.4 | — | — |
| Anti-oxidants | 0.05 | 0.05 | 0.05 | 0.05 | 0.05 | 0.05 |
| Sweetener | 5.0 | 5.0 | 5.0 | 5.0 | 5.0 | 5.0 |
| Microbial preservative | 0.05 | 0.05 | 0.05 | 0.05 | 0.05 | 0.05 |
| Purified water | To 100 | To 100 | To 100 | To 100 | To 100 | To 100 |

All emulsion cannabinoid composition of table 8 passed the centrifugation stability test, were translucent, with max particle size of below about 1 micron and mean particle size from 50 nm to 200 nm.

### Example 9. Concentrated Emulsifiable Composition Comprising a Cannabinoid and Terpenes

A mixture of 2.0 gram of pure CBD was mixed with 1.5 grams of mixture of equal parts of myrcene:caryphyllene:pinene, 4.0 grams of capric/caprylic triglyceride oil, 1.5 grams of sucrose distearate, 1.5 grams of PEG-35 castor oil, and 1.5 grams of tocopheryl PEG1000 succinate. The mixture was heated shortly to about 80° C. and homogenized until mixture was homogeneous.

### Example 10. Oral Absorption of CBD in Rats

A concentrate of example 9 was diluted with hot water of about 40° C. before administration to the rats to obtain CBD concentration of 12 mg/ml and to obtain the cannabinoid oil-in-water emulsion. The emulsion was administered in gavage to the stomach of Sprague drawly rats weighing 220 gr to 250 gr at 12 mg/kg. Blood sample was obtained at various time points after the oral administration and plasma separated. The CBD concentration in the plasma was tested by LC-MS and the area under the curve calculate. Group one was administered with 12 mg/kg of pure CBD dissolved in “solvent” propylene glycol:ethanol 1:1 solution. Group two received the test formulation.

TABLE 10
Area Under the Curve (AUC) and Cmx of CBD in rat's plasma

| Test item | AUC ng/ml/hour | Cmax ng/ml |
|---|---|---|
| Group one: | 90 +/− 12 | 38 |
| 12 mg/kg of CBD dissolved in propylene | | |
| glycol:ethanol | | |
| Group two: | 392 +/− 32 | 133 |
| 12 mg/kg of diluted concentrate of | | |
| example 9 (MCT oil) | | |
| Group three | 340 +/− 28 | 124 |
| 12 mg/kg of example 8B (Olive oil) | | |
| Group four | 335 +/− 24 | 118 |
| 12 mg/kg of example 8C (Sesame oil) | | |

### Example 11

TABLE 11
Mean particle size measurement (Malvern nanosizer ™) of stable
emulsion compositions comprising various emulsifiers combinations

| Batch number | 317 | 417 | 517 | 617 | 717 | 817 | 917 |
|---|---|---|---|---|---|---|---|
| THC | 0.2 | 0.2 | 0.2 | 0.2 | 0.2 | 0.2 | 0.2 |
| MCT | 3.8 | 3.8 | 3.8 | 3.8 | 3.8 | 9.8 | 3.8 |
| TPGS | 0.8 | 1.2 | 0 | 2 | 2 | 2.5 | 1.2 |
| Sucrose ester | 0.8 | 1.2 | 1.2 | 0 | 2 | 2.5 | 0 |
| Cromophor | 0.8 | 1.2 | 1.2 | 2 | 0 | 3 | 1.2 |
| EL35 | | | | | | | |
| Eucaliptus oil | 0.5 | 0.5 | 0.5 | 0.5 | 0.5 | 0.5 | 0.5 |
| Tween 80 | 0 | 0 | 0 | 0 | 0 | 0 | 1.2 |
| Water ad | 93.1 | 91.9 | 93.1 | 91.5 | 91.5 | 81.5 | 91.9 |
| Nanosizer | | | | | | | |
| results | | | | | | | |
| Pick 1 nm | 83 | 54 | 85 | 84 | 16.5 | 114 | 94 |
| Pick 2 nm | | | 4032 | 1360 | 104 | 1520 | |
| Pick 3 nm | | | | | 1484 | | |

### Example 12

Formulation 6A is a gel and was prepared by adding alkyl acrylate cross copolymer (Pemulene™ TR2) and raising the pH to above about pH 5.5 with a solution of 0.1N sodium hydroxide. The gel is viscous but pour-able, and the viscosities is about 3,000 centipoise, The viscosity of the gel can be adjusted as needed for medication by changing the amount of the alkyl acrylate croeee polymer.

### Example 13

Formulations 6A, 6F, 6G and 6H were subjected to freeze and thaw cycle, stored a −15° C. for 24 hours and thawed at ambient temperature and examined for presence of separation, creaming or color change and maximal particle size. Compositions 6A, 6F and 6H passed the freeze thaw test and did not separate or cream and the maximal particle size did not changed as examined by light microscope.

### Example 14

TABLE 12
High strength and stable sub-micron emulsion compositions comprising high
cannabinoids concentration.

| | Formula # | | | | | | |
|---|---|---|---|---|---|---|---|
| | 9A | 9 | 9C | 9D | 9E | 9F | 10F |
| Ingredient | % W/W | % W/W | % W/W | % W/W | % W/W | % W/W | % W/W |
| CBD | 4.0 | 4.0 | 6.0 | 6.0 | 8.0 | 8.0 | 10.0 |
| Terpenes mixture | — | — | — | 0.1 | 0.1 | 0.1 | 0.1 |
| Capric/caprylic triglyceride | 4.0 | — | — | 4.0 | — | — | — |
| Olive oil | — | 4.0 | — | — | 4.0 | — | — |
| Sesame oil | — | — | 6.0 | — | — | 6.0 | 6.0 |
| Hydrogenated castor oil | 4.0 | 4.0 | 4.0 | | | | |
| Behenyl alcohol | — | — | — | 4.0 | 4.0 | 4.0 | 4.0 |
| Tocopheryl PEG 1000 | 1.5 | 1.5 | 1.5 | 1.5 | 1.5 | 1.5 | 2.0 |
| succinate | | | | | | | |
| PEG-35 Castor Oil | 0.6 | 0.6 | 0.6 | 0.6 | 0.6 | 0.6 | 0.8 |
| Polyclycerol-3 dioleate | 0.6 | 0.6 | 0.6 | 0.6 | 0.6 | 0.6 | 0.8 |
| Pemulene TR2 | — | 0.5 | — | 0.5 | — | 0.5 | 0.5 |
| Sucralose | 2.0 | 2.0 | 2.0 | 2.0 | 2.0 | 2.0 | 2.0 |
| Glycynthizic Acid. | 0.5 | 0.5 | 0.5 | 0.5 | 0.5 | 0.5 | 0.5 |
| (Magnasweet ® 110) | | | | | | | |
| Glycerin | — | 20.0 | — | 20.0 | — | 20.0 | 20.0 |
| Anti-oxidants | 0.05 | 0.05 | 0.05 | 0.05 | 0.05 | 0.05 | 0.05 |
| Buffer as needed to pH 6.5 | q.s. | q.s. | q.s. | q.s. | q.s. | q.s. | q.s. |
| Flavore raspberry syrup | 5.0 | 5.0 | 5.0 | 5.0 | 5.0 | 5.0 | 5.0 |
| Microbial preservative | 0.05 | 0.05 | 0.05 | 0.05 | 0.05 | 0.05 | 0.05 |
| Sucrose syrup | 40.0 | 20.0 | 60.0 | 40.0 | 70.0 | 30.0 | 30.0 |
| Purified water | To 100 | To 100 | To 100 | To 100 | To 100 | To 100 | To 100 |

Formulations of example 14 do not separate upon centrifugation of 5 min under 5,000 RPM and are essentially submicron as tested by light microscope by the brownnian motion of the particles with few non sub micron droplets whereas the largest particle size up to about 5 microns.

1. A stable oil-in-water submicron emulsion oral cannabinoid composition, comprising from about 0.05% w/w to about 10% w/w of at least one cannabinoid in a pharmaceutically acceptable carrier, from about 1% w/w to about 10% w/w of at least one triglyceride oil, from about 1% w/w to about 10% w/w of at least two emulsifiers, of which one having an HLB value from about 10 to about 16 and the other(s) having an HLB from about 2 to about 8, from about 1% w/w to about 40% w/w of at least one taste-enhancing excipient and from about 1% w/w to about 60% w/w of water,
wherein the composition is essentially free of bitter taste, and
wherein the ratio between the at least one cannabinoid and the at least two emulsifiers is at least 1:5, and
wherein the emulsion oil phase comprises not more than 10% by weight of the composition and
wherein the composition is essentially free of phospholipids, liposomes and/or micelles.

2. The composition of claim 1, wherein the emulsion is stable at ambient temperature for at least 12-24 months and does not separate or cream under accelerated conditions.

3. The composition of claim 1, wherein the at least one taste-enhancing excipient is selected from sucrose, glucose, sucralose, glycerin, aspartame, cyclamate, stevia, erythritol, xylitol, acesulfam, honey, sucrose syrup, maple syrup, raspberry syrup, glycyrrhizin, glycyrrhitinic acid, glycyrrhizinate salts, Glycyrrhiza glabra extracts and combinations thereof.

4. The composition of claim 3, wherein the al least one taste-enhancing excipient is comprised of about 2% w/w of sucralose, about 0.5% w/w of liquid ammonium glycyrrhizinate (Magnasweet® 110), and a syrup selected from sucrose syrup, maple syrup, raspberry syrup or artificial syrup to make up to 100% by weight.

5. The composition of claim 1, wherein the at least one cannabinoid is at a high strength, and wherein a teaspoon of the syrup composition comprises at least 100 mg, at least 200 mg or at least 500 mg of a cannabinoid.

6. The composition of claim 1, wherein the ratio by weight of the at least one cannabinoid and the ratio of the at least one triglyceride oil to the at least two emulsifiers is from 1:2 to about 10:1 and wherein the total cannabinoids amount by weight is above its solubility in the oil

7. The composition of claim 1, wherein the at least one cannabinoid is selected from the group consisting of a natural or synthetic cannabinoid or cannabinoid combination: selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof, cannabis oil, cannabis extract and combinations thereof.

8. The composition of claim 1, wherein the at least one triglyceride oil is selected from the group consisting of vegetable oil, corn oil, peppermint oil, canola oil, poppy seed oil, palm oil, soybean oil, hydrogenated soybean oil, sesame oil, rapeseed oil, sunflower seed oil, peanut oil, castor oil, hydrogenated castor oil, cottonseed oil, palm kernel oil, coconut oil, olive oil, borage oil and combinations thereof.

9. The composition of claim 1, wherein at least one of the at least two emulsifiers having a HLB value of from about 10 to about 16 is selected from the group consisting of sucrose ester, sucrose fatty acid esters, sucrose stearate, sucrose distearate, sucrose palmitate, sucrose laurate, sucrose polystearate, polysorbate, polysorbate 80, oleoyl polyoxyl glycerides, polyoxyl hydrogenated castor oil, tocopherol polyethylene glycol 1000 succinate, polyoxyl glycerides, polyglyceryl fatty acid ester, and polymeric emulsifier Acrylates/C 10-30 Alkyl Acrylate Cross-Polymer and combinations thereof.

10. The composition of claim 1, wherein the at least one emulsifier having HLB from about 2 to about 8 is selected from the group consisting of sorbitan fatty acid esters, sorbitan monooleate, sorbitan stearate, sorbitan oleate, sucrose ester, sucrose di and tri stearate, polyglyceryl fatty acid esters, polyglyceryl-3 dioleate, fatty acids esters and ethers, steareat-2, oleth-2, ceteth-2, salts thereof, derivatives thereof, and combinations thereof.

11. The composition of claim 1, wherein the at least one cannabinoid exhibits an improved oral bioavailability at least about 100% higher than the oral bioavailability of same cannabinoid, when dissolved in an organic solvent or a mixture of organic solvents.

12. The composition of claim 1, wherein the ratio between the at least one cannabinoid and the at least two emulsifiers is from about 1:4 to 10:1, from about 1:2 to about 5:1 or from about 1:1 to about 3:1.

13. The composition of claim 1, wherein further comprising from about 0.1% w/w to about 5% w/w of at least one stabilizing or suspending agent.

14. A dosage form comprising the composition of claim 1, wherein said dosage form is selected from an emulsion, a syrup, a liquid, a pudding, a gel and wherein administered with a spoon, a dropper or other volume measuring device or mixed with nutrients or foods.

15. A kit for the treatment of geriatric syndrome and improvement of elderly and neurological patient's wellbeing, epilepsy, PTSD, anxiety, schizophrenia, Parkinson disease and autoimmune diseases that are also suffering from insomnia and improvement of their wellbeing comprising a day composition and a night composition according to claim 1, comprising a day composition comprising a. at least about 10 mg cannabis extract or at least one cannabinoid or derivatives thereof having a CBD:THC ratio of about 10 to about 200 and b. at least about 1 mg of at least one alerting terpene.
and a night composition, comprising:
a. at least about 1 mg to about 20 mg of cannabis extract or at least one cannabinoid or derivatives thereof and
b. at least about 1 mg of at least one sedative terpene.

16. A method of treatment for inducing an alerting response by administration to a mammal subject in need thereof of a therapeutically effective amount of the composition of claim 1, wherein said composition comprising at least one cannabinoid selected from at least about 1 mg of cannabidiol, a derivative thereof and combinations thereof and at least about 1 mg of at least one alerting terpene per one serving.

17. The method of claim 16, wherein the at least one alerting terpene is selected from limonene, alfa and beta pinene, orange terpenes, thymol, isoborneol, and isoeugenol or citronella or orange essential oils or lavender essential oil, caryophyllene, rosmarinus oil, citrus oil and combinations thereof.

18. A method of treatment for sedation and/or sleep inducing in a mammal in need thereof, by administration at about one hour before retiring to sleep or as needed of a therapeutically effective amount of the composition of claim 1, wherein comprising from about 1 mg to about 10 mg of at least one sedating cannabinoid and from about 0.5 mg to about 5 mg of at least one sedating terpene per unit serving.

19. The method of claim 18, wherein the sedating cannabinoid is selected from Tetrahudrocannabinol (THC), Cannabinol (CBN) and combinations thereof, and the sedating terpene is selected from myrcene, linalool, linalyl acetate, alfa terpineol and citronellal, sandalwood, lavender, valerian, neroli essential oils and combinations thereof.

20. The composition of claim 1 wherein the at least one cannabinoid is CBD.
